(19) United States
(12) Patent Application Publication
(10) Pub. No.: US 2025/0009326 A1
(43) Pub. Date: Jan. 9, 2025

(54) HEART SOUND ACQUISITION DEVICE, HEART SOUND ACQUISITION SYSTEM, HEART SOUND ACQUISITION METHOD, AND PROGRAM

(71) Applicant: TERUMO KABUSHIKI KAISHA, Tokyo (JP)

(72) Inventors: Tomonori HATTA, Hadano-shi (JP); Takayuki UCHIDA, Hiratsuka city (JP); Ryo ICHIKAWA, Atsugi-shi (JP)

(73) Assignee: TERUMO KABUSHIKI KAISHA, Tokyo (JP)

(21) Appl. No.: 18/891,249

(22) Filed: Sep. 20, 2024

Related U.S. Application Data
(63) Continuation of application No. PCT/JP2023/007699, filed on Mar. 1, 2023.

(30) Foreign Application Priority Data
Mar. 22, 2022 (JP) 2022-046115

(51) Int. Cl. A61B 7/04; A61B 5/021; A61B 5/24; A61B 90/00

(52) U.S. Cl. CPC A61B 7/04; A61B 5/021; A61B 5/24; A61B 90/08; A61B 2090/0808; A61B 2560/0406; A61B 2560/0462; A61B 2562/0247

(57) ABSTRACT

A heart sound acquisition device that includes a main body, a movable member, and a biasing portion. The movable member includes a first sensor that is brought into contact with a human body to acquire a heart sound. The biasing portion biases the movable member so that the first sensor presses the human body during acquisition of the heart sound. The movable member is movable with respect to the main body in a pressing direction in which the first sensor presses the human body, and the main body includes an adjustment portion that adjusts a biasing force of the biasing portion on the movable member.

### CROSS-REFERENCES TO RELATED APPLICATIONS

This application is a continuation of International Application No. PCT/JP2023/007699 filed on Mar. 1, 2023, which claims priority to Japanese Application No. 2022-046115 filed on Mar. 22, 2022, the entire content of both of which is incorporated herein by reference.

### TECHNOLOGICAL FIELD

The present disclosure relates to a heart sound acquisition device, a heart sound acquisition system, a heart sound acquisition method, and a program.

### BACKGROUND DISCUSSION

A patient with a chronic disease such as heart failure and respiratory diseases has a risk of deterioration in his/her condition even after discharge from a hospital, and thus it is important to continuously measure his/her vital signs. It is known that heart sounds include information suggesting heart diseases such as valvular disease and angina pectoris or respiratory diseases such as chronic obstructive pulmonary disease (COPD), and in recent years, it is also desired to perform measurement at home. In order to acquire an appropriate heart sound, it is necessary to place a sensor for measuring the heart sound at an accurate position and apply an appropriate pressure.

Conventionally, as a device for acquiring heart sounds at home, there has been proposed a stethoscope including a chest piece incorporating a microphone, the stethoscope including a sensor that detects contact and that is disposed at a contact portion of the chest piece with a human body (see, for example, Japanese Patent Application Publication No. 2017-170112 A).

However, a conventional device for measuring heart sounds used at home involves difficulty in pressing a sensor for acquiring heart sounds against a patient with an appropriate pressure. Therefore, in a case where a patient, a family member of the patient, or the like who is not a medical worker tries to measure heart sounds, a measurement result with good reproducibility may not be obtained.

### SUMMARY

A heart sound acquisition device, a heart sound acquisition system, a heart sound acquisition method, and a program with which it is possible to easily and satisfactorily acquire a heart sound with high reproducibility without expert knowledge.

A heart sound acquisition device as one aspect of the present disclosure includes: a main body; a movable member including a first sensor that is brought into contact with a human body to acquire a heart sound; and a biasing portion that biases the movable member to allow the first sensor to press the human body when the heart sound is acquired, wherein the movable member is movable with respect to the main body in a pressing direction that is a direction in which the first sensor presses the human body, and the main body includes an adjustment portion that adjusts a biasing force of the biasing portion on the movable member.

As one aspect, the biasing portion is engaged with the main body at a first end along the pressing direction, is engaged with the movable member at a second end closer to the first sensor with respect to the first end, and applies a force in a direction of increasing a distance between the first end and the second end.

As one aspect, the biasing portion includes at least one of an elastic body or a magnetic body.

As one aspect, the main body includes: a first member; and a second member that is located on a side of the first member opposite to a side on which the first sensor is mounted, the second member including a first movement restriction portion engaged with the first end of the biasing portion, and the second member includes the adjustment portion configured to adjust a position with respect to the first member in the pressing direction.

As one aspect, the main body includes a fixing member that fixes the adjustment portion so as to fix the biasing force.

As one aspect, the main body includes a second movement restriction portion that restricts movement of the movable member in the pressing direction by interfering with the movable member.

As one aspect, the main body includes a cover that comes into contact with the human body around the first sensor during acquisition of the heart sound.

As one aspect, the main body includes a cover that comes in contact with the human body around the first sensor during acquisition of the heart sound, the cover including the adjustment portion configured to adjust a position with respect to the second movement restriction portion in the pressing direction.

As one aspect, the heart sound acquisition device further includes a second sensor for acquiring biological information other than a heart sound on a surface of the cover to be in contact with the human body.

As one aspect, the heart sound acquisition device further includes an adhesive member that is detachably affixed to the human body on a surface of the cover to be in contact with the human body.

As one aspect, the main body includes at least one of a protrusion including a sliding surface for movably supporting the movable member in the pressing direction or a ball bearing.

As one aspect, the heart sound acquisition device further includes a mark used for relatively positioning the main body and the movable member in the pressing direction, the mark being attached on at least one of the main body or the movable member.

A heart sound acquisition system as one aspect of the present disclosure includes: a heart sound acquisition unit including a main body, a movable member including a first sensor that is brought into contact with a human body to acquire a heart sound, and a biasing portion that biases the movable member to allow the first sensor to press the human body when the heart sound is acquired, the movable member being movable with respect to the main body in a pressing direction that is a direction in which the first sensor presses the human body, the main body including an adjustment portion that adjusts a biasing force of the biasing portion on the movable member; and a control unit that determines an acquisition state of the heart sound on the basis of data indicating the heart sound acquired from the first sensor. As one aspect, the heart sound acquisition system further includes a

storage unit that stores a reference waveform of a heart sound measured in advance, wherein the data indicating the heart sound includes a measurement waveform of the heart sound, and the control unit determines the acquisition state of the heart sound by comparing the reference waveform with the measurement waveform.

As one aspect, the control unit determines the acquisition state of the heart sound on the basis of similarity between the reference waveform and the measurement waveform.

As one aspect, the heart sound acquisition system further includes an output unit, wherein the control unit causes the output unit to display at least one of an error or a guidance when determining that the reference waveform and the measurement waveform are not similar to each other.

As one aspect, the heart sound acquisition unit includes a third sensor that measures a pressure applied by the first sensor to press the human body.

As one aspect, the heart sound acquisition system further includes a storage unit that stores a reference pressure that is set in advance for acquiring a heart sound, wherein, when the pressure measured by the third sensor is not within a predetermined range based on the reference pressure, the control unit causes the output unit to display at least one of an error or a guidance.

As one aspect, the main body includes, as the adjustment portion, a pressure adjustment mechanism that adjusts pressure applied by the first sensor to press the human body, and the control unit adjusts the pressure by the pressure adjustment mechanism when determining that the reference waveform and the measurement waveform are not similar to each other.

As one aspect, the main body includes, as the adjustment portion, a pressure adjustment mechanism that adjusts pressure applied by the first sensor to press the human body, the data indicating the heart sound includes a measurement waveform of the heart sound, and the control unit adjusts the pressure by the pressure adjustment mechanism to maximize an amplitude of the measurement waveform.

As one aspect, the heart sound acquisition unit includes a third sensor that measures a pressure applied by the first sensor to press the human body.

As one aspect, the heart sound acquisition system further includes a storage unit that stores a reference pressure that is set in advance for acquiring a heart sound, wherein the control unit adjusts the pressure by the pressure adjustment mechanism to set the pressure measured by the third sensor to the reference pressure as an initial value of the pressure.

As one aspect, the biasing portion is engaged with the main body at a first end along the pressing direction, is engaged with the movable member at a second end closer to the first sensor with respect to the first end, and applies a force in a direction of increasing a distance between the first end and the second end, and the pressure adjustment mechanism adjusts a position of the main body engaged with the first end in the pressing direction.

As one aspect, the biasing portion includes a balloon that presses the movable member in the pressing direction, and the pressure adjustment mechanism adjusts pressure exerted on the movable member by the balloon by supplying air or discharging air into or from the balloon.

As one aspect, the control unit acquires biological information other than a heart sound from another measurement device, and estimates a cardiac function-related parameter on the basis of the heart sound acquired from the first sensor and the biological information.

As one aspect, the other measurement device includes at least one of an electrocardiograph or a blood-pressure meter.

As one embodiment, the cardiac function-related parameter includes an intracardiac pressure.

A heart sound acquisition method as one aspect of the present disclosure is a heart sound acquisition method executed by a control unit of a computer, the method comprising: acquiring a heart sound from a heart sound acquisition unit that includes a main body, a movable member including a first sensor that is brought into contact with a human body to acquire a heart sound, and a biasing portion that biases the movable member to allow the first sensor to press the human body when the heart sound is acquired, the movable member being movable with respect to the main body in a pressing direction that is a direction in which the first sensor presses the human body, the main body including an adjustment portion that adjusts a biasing force of the biasing portion on the movable member; and determining an acquisition state of the heart sound on the basis of a measurement waveform of the heart sound.

A non-transitory computer-readable storage medium as one aspect of the present disclosure stores a program that causes a processor to execute: acquiring a heart sound from a heart sound acquisition unit that includes a main body, a movable member including a first sensor that is brought into contact with a human body to acquire a heart sound, and a biasing portion that biases the movable member to allow the first sensor to press the human body when the heart sound is acquired, the movable member being movable with respect to the main body in a pressing direction that is a direction in which the first sensor presses the human body, the main body including an adjustment portion that adjusts a biasing force of the biasing portion on the movable member; and determining an acquisition state of the heart sound on the basis of a measurement waveform of the heart sound.

According to the heart sound acquisition device, the heart sound acquisition system, the heart sound acquisition method, and the program of the present disclosure, it is possible to adjust the pressure applied by the first sensor to press the human body by adjusting a biasing force of the biasing portion on the movable member. Thus, a heart sound can be easily acquired with high reproducibility without expert knowledge.

### BRIEF DESCRIPTION OF THE DRAWINGS

FIG. 1 is a perspective view of a heart sound acquisition device according to a first embodiment.

FIG. 2 is a cross-sectional view of the heart sound acquisition device in FIG. 1.

FIG. 3 is a cross-sectional view of a heart sound acquisition device according to a second embodiment.

FIG. 4 is a diagram illustrating another configuration example of a movable member and a biasing portion of the heart sound acquisition device.

FIG. 5 is a perspective view of a heart sound acquisition device according to a third embodiment.

FIG. 6 is a cross-sectional view of the heart sound acquisition device in FIG. 5.

FIG. 7 is a cross-sectional view of a heart sound acquisition device according to a fourth embodiment.

FIG. 8 is a cross-sectional view of a heart sound acquisition device according to a fifth embodiment.

FIG. 9 is a cross-sectional view of a heart sound acquisition device according to a sixth embodiment.

FIG. 10 is a cross-sectional view of a heart sound acquisition device according to a seventh embodiment.

FIG. 11 is a cross-sectional view of a heart sound acquisition device according to an eighth embodiment.

FIG. 12 is a cross-sectional view of a heart sound acquisition device according to a ninth embodiment.

FIG. 13 is a cross-sectional view of a heart sound acquisition unit according to a tenth embodiment.

FIG. 14 is a cross-sectional view of a heart sound acquisition unit according to an eleventh embodiment.

FIG. 15 is a block diagram illustrating a schematic configuration of a heart sound acquisition system according to a twelfth embodiment.

FIG. 16A is a part of a flowchart illustrating a procedure for acquiring heart sound data using the heart sound acquisition system in FIG. 15.

FIG. 16B is another part of the flowchart illustrating the procedure for acquiring heart sound data using the heart sound acquisition system in FIG. 15.

FIG. 17 is a block diagram illustrating a schematic configuration of a heart sound acquisition system according to a thirteenth embodiment.

FIG. 18 is a flowchart illustrating a procedure for acquiring a reference waveform stored in a storage unit illustrated in FIG. 17.

FIG. 19 is a flowchart illustrating a procedure for acquiring heart sound data using the heart sound acquisition system in FIG. 17.

FIG. 20 is a block diagram illustrating a schematic configuration of a heart sound acquisition system according to a fourteenth embodiment.

FIG. 21 is a flowchart illustrating a procedure for acquiring heart sound data using the heart sound acquisition system in FIG. 20.

FIG. 22 is a block diagram illustrating a schematic configuration of a heart sound acquisition system according to a fifteenth embodiment.

FIG. 23 is a flowchart illustrating a procedure for measuring and estimating a parameter related to a cardiac function using the heart sound acquisition system in FIG. 22.

### DETAILED DESCRIPTION

Set forth below with reference to the accompanying drawings is a detailed description of embodiments of a heart sound acquisition device, a heart sound acquisition system, a heart sound acquisition method, and a program. The drawings used in the following description are schematic. Shapes, ratios, and the like in the drawings do not necessarily coincide with actual ones.

### First Embodiment

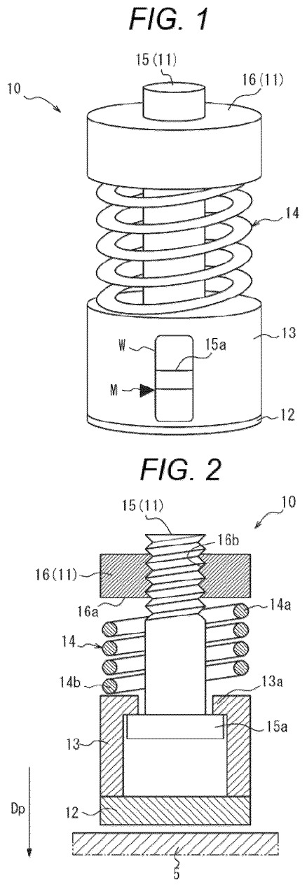

A heart sound acquisition device 10 according to the first embodiment includes a main body 11, a movable member 13 having a first sensor 12, and a biasing portion 14 as illustrated in the perspective view of FIG. 1 and the cross-sectional view of FIG. 2. The heart sound acquisition device 10 is a device that acquires a heart sound by bringing the first sensor 12 into contact with the chest of a human body 5 that is a heart sound acquisition site. The heart sound acquisition device 10 is assumed to be used at home by a user who does not have medical expertise, such as a subject himself/herself or a close relative of the subject. The heart sound acquisition device 10 may be used by a medical worker such as a doctor in a medical facility.

The first sensor 12 is a sensor that detects a sound including a heart sound. The first sensor converts a sound including a heart sound into, for example, an electric signal. The first sensor 12 can be selected from various sensors such as a condenser microphone that detects a change in capacitance between a diaphragm (diaphragm) and a back plate (electrode), a piezoelectric microphone using a piezoelectric element, and a dynamic microphone obtained by combining a permanent magnet and a coil. The first sensor 12 can output the detected heart sound to other components in the heart sound acquisition device 10 or to the outside of the heart sound acquisition device 10.

The first sensor 12 is not limited to a sensor that converts a sound including a heart sound into an electric signal. A mode in which the sound including a heart sound acquired by the first sensor 12 is not converted into an electric signal is also included. In that case, the heart sound may be transmitted to the doctor or other medical experts as the vibration of air or object.

The first sensor 12 is located at one end (lower side in FIG. 2) of the heart sound acquisition device 10 because the first sensor is brought into contact with the human body 5. In the following, the side (upper side in FIG. 2) opposite to the first sensor 12 of the heart sound acquisition device 10 is referred to as a first side. In addition, the first sensor 12 side of the heart sound acquisition device 10 (lower side in FIG. 2) is referred to as a second side. The second side is the human body 5 side when the heart sound acquisition device 10 is used.

The main body 11 is a portion at least a part of which can be held by a user. The main body 11 includes a first member 15 and a second member 16. The main body 11 may include not only the first member 15 and the second member 16 but also three or more members. The first member 15 is a substantially columnar member. A spiral groove is formed around an end of the first member 15 on the first side. A flange 15a extending outward from a columnar portion is disposed at an end of the first member 15 on the second side.

The second member 16 is a ring-shaped member having a hole through which the first member 15 passes in a central part. A spiral groove is formed in the hole of the second member 16. The second member 16 is rotatably and slidably screwed to the outside of the first member 15 around the end of the first member 15 on the first side. The second member 16 can be moved forward and backward in a pressing direction D_{P} along the first member 15 by rotating the second member 16 with respect to the first member 15.

A surface 16a of the second member 16 on the second side contacts a first end 14a that is an end of the biasing portion 14 on the first side to restrict the movement of the biasing portion 14 and the movement of the movable member 13 via the biasing portion 14 in the direction opposite to the pressing direction Dₚ.

The movable member 13 is movable in the pressing direction Dₚ within a limited range with respect to the main body 11. Here, the term “movable” is used in a relative sense. In a case where the movable member 13 is brought into contact with and fixed to the human body 5, movement of the movable member 13 with respect to the main body 11 means movement of the main body 11 with respect to the human body 5.

The movable member 13 has a cylindrical shape having a space into which a part of the first member 15 on the second side is inserted. A protrusion 13a extending to the inside of the cylinder is formed at an end of the movable member 13 on the first side. The protrusion 13a interferes with the flange 15a formed at the end of the first member 15 on the second side, so that the movement of the movable member 13 in the pressing direction Dₚ is restricted.

The movable member 13 may include a window W through which the position of the flange 15a of the first member 15 can be visually recognized as illustrated in FIG. 1. In addition, a mark M for aligning the flange 15a may be disposed beside the window W.

The biasing portion 14 is in contact with and is engaged with the surface 16a of the second member 16 directed to the second side at the first end 14a on the first side. Further, the biasing portion 14 is in contact with and is engaged with a surface of the protrusion 13a of the movable member 13 directed to the second side at the second end 14b. The biasing portion 14 generates a force in a direction of increasing a distance between the first end 14a and the second end 14b. Accordingly, the biasing portion 14 biases the movable member 13 so that the first sensor 12 presses the human body 5 during acquisition of the heart sound.

The biasing portion 14 can include, for example, at least one of an elastic body or a magnetic body. The elastic body can include a spring in a state of being compressed from a natural length. The magnetic body can include a magnet in which N poles or S poles are arranged to face each other. As the biasing portion 14, a member using compression of air, a member using electromagnetic force, a member using rubber, or the like can be further used.

The surface 16a of the second member 16 is a first movement restriction portion that restricts the movement of the biasing portion 14 and the movable member 13 located on the second side of the biasing portion 14 in the direction opposite to the pressing direction Dₚ. The flange 15a of the first member 15 is a second movement restriction portion that restricts the movement of the movable member 13 in the pressing direction Dₚ. The flange 15a prevents detachment of the movable member 13 from the first member 15.

Optionally, the heart sound acquisition device 10 may have an attachment means for continuously measuring a predetermined position of the human body 5. For example, the heart sound acquisition device 10 can be configured as a patch-type device in which the surface of the first sensor 12 that is to be in contact with the human body 5 is affixed to the skin of the human body 5. In addition, the heart sound acquisition device 10 may include a strap belt for fixing the first sensor 12 to the human body 5.

With the above-described configuration, the user of the heart sound acquisition device 10 can acquire the heart sound by holding the second member 16 in his/her hand and bringing the first sensor 12 into contact with the human body 5 to press the human body 5. During this operation, a pressure corresponding to the biasing force of the biasing portion 14 is applied to the human body 5. The biasing force generated by the biasing portion 14 changes depending on the distance between the first end 14a and the second end 14b of the biasing portion 14. For example, when the biasing portion 14 is a spring, a force proportional to the difference between the distance between the first end 14a and the second end 14b and the natural length of the spring can be generated.

The distance between the first end 14a and the second end 14b of the biasing portion 14 is determined by the positional relationship between both the first member 15 and the second member 16 (main body 11) and the movable member 13. Therefore, the user positions the flange 15a visually recognized through the window W disposed in the movable member 13 at a predetermined position according to the mark M disposed in the main body 11, thereby being capable of easily setting the pressure applied by the first sensor 12 to press the human body 5 to a predetermined pressure optimum for acquiring the heart sound. For example, when the subject visits a medical institution, the doctor may set the mark M at a position where the pressure applied by the first sensor 12 to press the human body 5 is optimum while auscultating the heart sound. When measuring the heart sound at home, the subject can use the mark M set by the doctor.

The method for positioning the main body 11 and the movable member 13 by the user is not limited to the above method. For example, the user may align the surface on the first side of the protrusion 13a and a mark disposed on the columnar portion of the first member 15 to perform positioning in the pressing direction Dₚ. Therefore, the window W is not always necessary. In addition, in the following description, the pressure applied by the heart sound sensor to press the human body may be referred to as pressing, and the value of the pressing may be referred to as a pressure value.

In addition, since the second member 16 is screwed into the first member 15, the position of the second member 16 in the pressing direction Dₚ with respect to the first member 15 can be displaced by rotating the second member 16 around the first member 15. When the second member 16 is displaced, the distance between the first end 14a and the second end 14b of the biasing portion 14 changes, so that the biasing force for biasing the movable member 13 by the biasing portion 14 changes. That is, the biasing force of the biasing portion 14 on the movable member 13 can be adjusted by adjusting the position of the second member 16. A threaded portion 16b of the second member 16 that adjusts the biasing force is an adjustment portion that adjusts the biasing force.

As described above, the heart sound acquisition device 10 according to the first embodiment can adjust the pressure applied by the first sensor 12 to press the human body 5 to a predetermined pressure value by adjusting the biasing force of the biasing portion 14 on the movable member 13. Thus, the user can easily acquire the heart sound with high reproducibility without having expert knowledge, and the accuracy of acquiring the heart sound can be improved.

### Second Embodiment

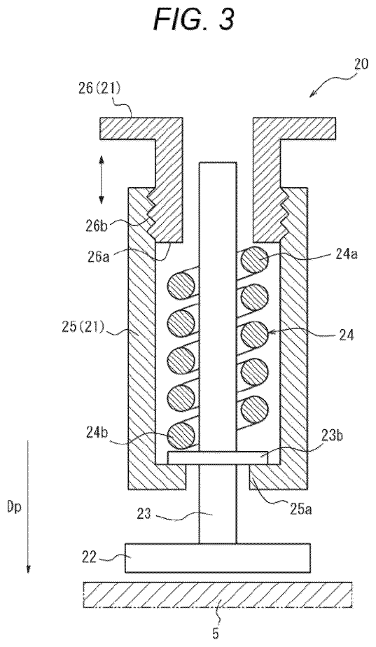

A heart sound acquisition device 20 according to the second embodiment will be described with reference to FIG. 3. Components identical or similar to those of the heart sound acquisition device 10 according to the first embodiment are denoted by reference numerals obtained by adding 10 to the reference numerals of the first embodiment. In the following, differences from the first embodiment will be described, and the description of contents overlapping with the first embodiment may be omitted.

The heart sound acquisition device 20 includes a main body 21, a movable member 23 having a first sensor 22, and a biasing portion 24. The first sensor 22 is a sensor that converts a sound including a heart sound into an electric signal like the first sensor 12 in the first embodiment.

The main body 21 includes a first member 25 and a second member 26. The first member 25 is a cylindrical member. A spiral groove is formed in an inner surface of the first member 25 in the vicinity of an end on the first side. An end of the first member 25 on the second side has a ring-shaped annular protrusion 25a extending inward from the inner surface of the cylindrical portion.

The second member 26 is a cylindrical member including a flange at an end on the first side. The second member 26 is rotatably and slidably screwed into the spiral groove formed on the inner surface of the first member 25. The second member 26 can be moved forward and backward in a pressing direction Dₚ by rotating the second member 26 with respect to the first member 25.

A surface 26a of the second member 26 on the second side is a first movement restriction portion that contacts a first end 24a that is an end of the biasing portion 24 on the first side to restrict the movement of the biasing portion 24 and the movement of the movable member 23 via the biasing portion 24 in the direction opposite to the pressing direction Dₚ.

The movable member 23 is a columnar member having a protrusion 23b extending outward. The first side of the movable member 23 extends into a space inside the cylindrical portions of the first member 25 and the second member 26. The protrusion 23b may have a rod shape or a disk shape extending in the radial direction from the movable member 23. The protrusion 23b interferes with the annular protrusion 25a formed at the end of the first member 25 on the second side, so that the movement in the pressing direction Dₚ can be restricted. The movable member 23 has the first sensor 22 at the end on the second side.

A mark for positioning the movable member 23 in use may be disposed in a section of the movable member 23 located outside the first member 25 on the second side. The user may position the movable member 23 by aligning the mark with the surface of the first member 25 on the second side.

The biasing portion 24 is in contact with and is engaged with the surface 26a of the second member 26 directed to the second side at the first end 24a on the first side. Further, the biasing portion 24 is in contact with and is engaged with the protrusion 23b of the movable member 23 at the second end 24b. The biasing portion 24 generates a force in a direction of increasing a distance between the first end 24a and the second end 24b. Accordingly, the biasing portion 24 biases the movable member 23 so that the first sensor 22 presses the human body 5 during acquisition of the heart sound. The biasing portion 24 can be configured similarly to the biasing portion 14 of the first embodiment.

The surface 26a of the second member 26 is a first movement restriction portion that restricts the movement of the biasing portion 24 and the movable member 23 located on the second side of the biasing portion 24 in the direction opposite to the pressing direction Dₚ. The annular protrusion 25a of the first member 25 is a second movement restriction portion that restricts the movement of the movable member 23 in the pressing direction Dₚ. The annular protrusion 25a prevents detachment of the movable member 23 from the first member 25.

With the above-described configuration, the user of the heart sound acquisition device 20 can acquire the heart sound by holding the second member 26 in his/her hand and bringing the first sensor 22 into contact with a heart sound acquisition site of the human body 5 to press the heart sound acquisition site. The user aligns the first member 25 and the movable member 23 using a mark or the like disposed on the movable member 23 to acquire a heart sound. The user can rotate the second member 26 to move the surface 26a forward or backward in the pressing direction Dₚ for positioning as with the heart sound acquisition device 10 according to the first embodiment. Thus, the pressure applied by the first sensor 22 to press the human body 5 can be adjusted to a predetermined pressure value.

In addition, in the heart sound acquisition device 20 according to the second embodiment, the displacement of the movable member 23 in the direction orthogonal to the pressing direction Dₚ is suppressed by the second member 26 on the first side. In addition, displacement of the movable member 23 in the direction orthogonal to the pressing direction Dₚ is suppressed by the annular protrusion 25a of the first member 25 on the second side. Therefore, the heart sound acquisition device 20 can have less rattling in the direction orthogonal to the pressing direction Dₚ than the heart sound acquisition device 10 according to the first embodiment, and can easily press the first sensor 22 perpendicularly to the surface of the human body 5.

Furthermore, in the heart sound acquisition device 20 according to the second embodiment, the biasing portion 24 including a spring or the like is not exposed to the outside, and thus may be superior in appearance to the heart sound acquisition device 10 according to the first embodiment. Furthermore, the heart sound acquisition device 20 is excellent in safety because there is no risk that a finger is caught by the biasing portion 24.

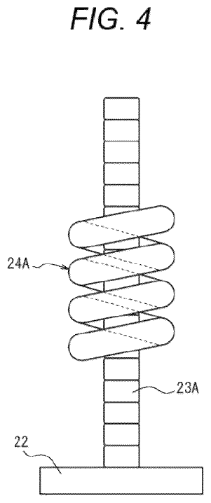

Note that the heart sound acquisition device 20 according to the second embodiment can employ a member obtained by integrating a movable member 23A and a biasing portion 24A as illustrated in FIG. 4. For example, the entire movable member 23A can be formed of a spring. A part of the spring has a larger outer periphery and a loose helix with a wide interval, and another part has a smaller outer periphery and a tight helix with a narrow interval. Thus, the region with a loose helix has relatively large elasticity, and the region with a tight helix has a rigid structure with relatively small elasticity.

Due to the member obtained by integrating the movable member 23 and the biasing portion 24 as described above, a section of the spring having a large outer periphery directly comes into contact with the annular protrusion 25a of the first member 25, so that the protrusion 23b becomes unnecessary when the member is used for the heart sound acquisition device 20.

### Third Embodiment

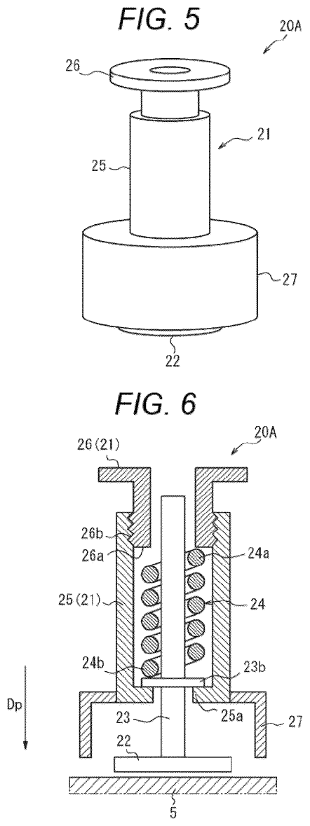

A heart sound acquisition device 20A according to the third embodiment will be described with reference to the perspective view of FIG. 5 and the cross-sectional view of FIG. 6. The heart sound acquisition device 20A is common to the heart sound acquisition device 20 according to the second embodiment in many points, and thus, the identical or similar components to those of the heart sound acquisition device 20 according to the second embodiment are denoted by the same reference numerals as those of the second embodiment. In the following, differences from the heart sound acquisition device 20 according to the second embodiment will be described, and the description of contents overlapping with the second embodiment will be omitted as appropriate.

The heart sound acquisition device 20A according to the third embodiment is obtained by adding a cover 27 disposed on the second side of the first member 25 of the heart sound acquisition device 20 according to the second embodiment as a part of the first member 25. The cover 27 can include, for example, a ring-shaped disk portion connected inside to the annular protrusion 25a having an annular shape, and a cylindrical portion extending in the pressing direction Dₚ from the outer periphery of the ring-shaped disk portion. The movable member 23 extends inside the cover 27. The shape of the cover 27 is not limited to a ring-shaped disk portion and a cylindrical portion extending in the pressing direction Dₚ. For example, the cover 27 may be a hemispherical member extending from the cylindrical portion of the first member 25 to the second side.

When the heart sound is not measured, the surface of the first sensor 22 to be in contact with the human body 5 is located on the second side with respect to an end of the cover 27 on the second side. When acquiring the heart sound, the user pushes the main body 21 in the pressing direction Dₚ to bring the end of the cover 27 on the second side into contact with the human body around the first sensor 22. In this way, the heart sound acquisition device 20A can help prevent the user from excessively pressing the first sensor 22 against the human body 5.

The shape and size of the cover 27 define the relative position of the first sensor 22 with respect to the first member 25 at the time of acquiring the heart sound. Accordingly, the distance between the first end 24a and the second end 24b of the biasing portion 24 is defined, and thus, the biasing force generated by the biasing portion 24 can be restricted. Therefore, with the heart sound acquisition device 20A, the user can press the first sensor 22 against a heart sound acquisition site of the human body 5 with a preset pressure value without visually positioning the main body 21 and the movable member 23 in the pressing direction Dₚ. Furthermore, since the user does not visually perform positioning, reproducibility of measuring the heart sound can be improved.

### Fourth Embodiment

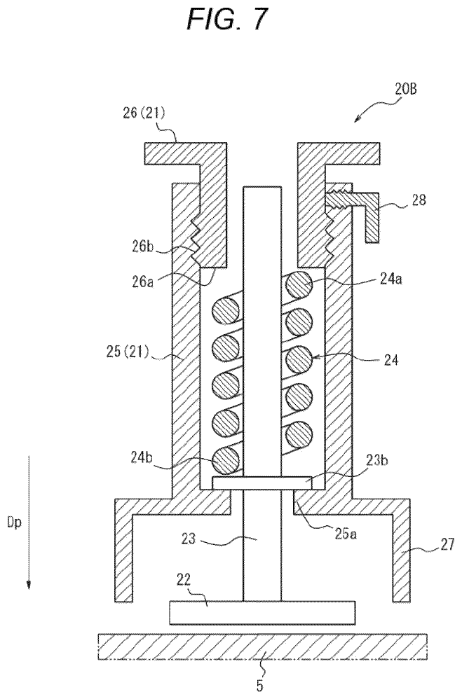

A heart sound acquisition device 20B according to the fourth embodiment will be described with reference to FIG. 7. The heart sound acquisition device 20B is obtained by adding a fixing member 28 to the heart sound acquisition device 20A according to the third embodiment. The other configurations of the heart sound acquisition device 20B are the same as those of the heart sound acquisition device 20A according to the third embodiment, and thus, the same components are denoted by the same reference numerals as those of the heart sound acquisition device 20A according to the third embodiment, and the description of the same components as those of the heart sound acquisition device 20A will be omitted.

The fixing member 28 fixes a first member 25 and a second member 26 of a main body 21 to each other. The fixing member 28 can be, for example, a member having an external thread at a tip of the fixing member 28. The fixing member 28 is screwed into a screw hole formed in a side surface of the cylindrical portion of the first member 25, passes through the screw hole, and presses the second member 26. With this configuration, when the fixing member 28 is tightened, the second member 26 is fixed to the first member 25 by frictional force with the fixing member 28. As a result, the fixing member 28 fixes the position of a threaded portion 26b of the second member 26 serving as the adjustment portion. The fixation of the second member 26 by the fixing member 28 can be released by rotating the fixing member 28 and retracting the same along the screw hole of the first member 25. The fixing member 28 is not limited to a member having an external thread. The fixing member 28 may have any shape as long as it can fix the first member 25 and the second member 26 to each other.

With the configuration described above in the fourth embodiment, after rotating the second member 26 to adjust the pressure applied by a first sensor 22 to press a heart sound acquisition site of a human body 5, the user can hold the position of the second member 26 in the pressing direction Dₚ using the fixing member 28. Therefore, according to the heart sound acquisition device 20B of the fourth embodiment, it is easier to maintain the pressure for pressing the human body 5 by the first sensor 22 after the adjustment so as not to change.

In the fourth embodiment, the second member 26 may not have a structure that adjusts the position in the pressing direction Dₚ by rotation with respect to the first member 25, as long as the fixing member 28 can fix and position the second member 26 with respect to the first member 25. For example, the second member 26 may be configured to be slidable in the pressing direction Dₚ with respect to the first member 25 and to be fixed to the first member 25 with the fixing member 28 at any position.

### Fifth Embodiment

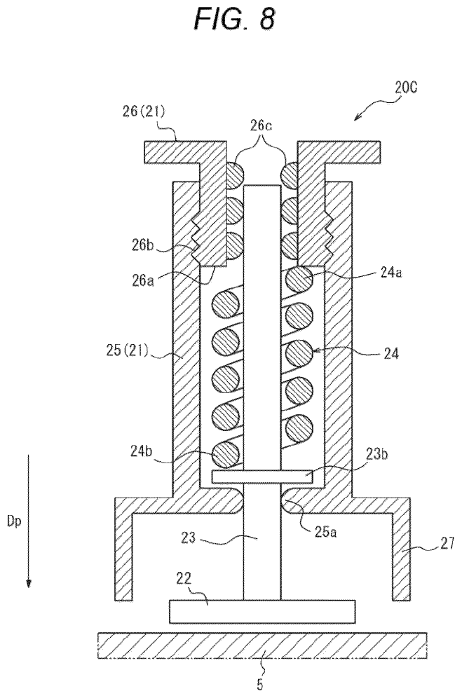

A heart sound acquisition device 20C according to the fifth embodiment will be described with reference to FIG. 8. The heart sound acquisition device 20C is obtained by adding a protrusion or a ball bearing serving as a guide for supporting the movable member 23 to the heart sound acquisition device 20A according to the third embodiment. The other configurations of the heart sound acquisition device 20C are the same as those of the heart sound acquisition device 20A according to the third embodiment, and thus, the same components are denoted by the same reference numerals as those of the third embodiment, and the description of the same components of the heart sound acquisition device 20A will be omitted as appropriate.

The heart sound acquisition device 20C is configured such that an inner end of an annular protrusion 25a of a first member 25 has a protrusion having a smooth curved surface, and comes in sliding contact with the side surface of a columnar portion of a movable member 23 as illustrated in FIG. 8. In addition, in the heart sound acquisition device 20C, protrusions 26c each having a sliding surface in sliding contact with the side surface of the columnar portion of the movable member 23 are disposed on the inner surface of a cylindrical portion of a second member 26. Thus, the movable member 23 is supported so as to be movable forward or backward in the pressing direction Dₚ. Instead of the annular protrusion 25a and the protrusions 26c, a ball bearing in contact with the movable member 23 may be disposed on the first member 25 and the second member 26.

The heart sound acquisition device 20C according to the fifth embodiment is configured as described above, whereby the movable member 23 can be smoothly moved forward and backward in the pressing direction Dₚ. As a result, it is possible to reduce the possibility that the pressure applied by a first sensor 22 to press a human body 5 is affected by factors such as the movable member 23 getting stuck with a main body 21 or the generation of frictional force between the movable member 23 and the main body 21.

### Sixth Embodiment

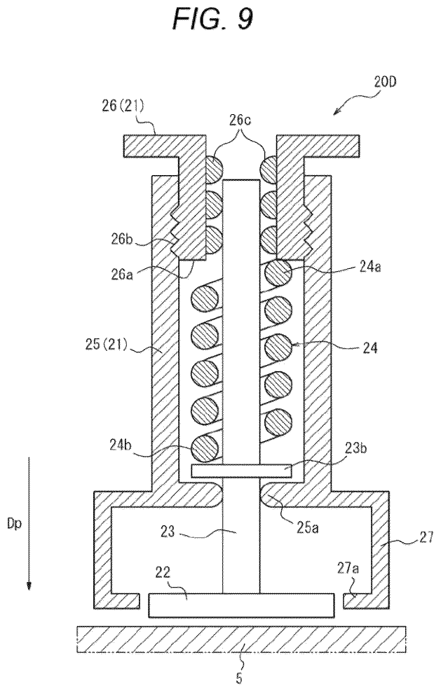

A heart sound acquisition device 20D according to the sixth embodiment will be described with reference to FIG. 9. The heart sound acquisition device 20D is obtained by adding a pushing control portion 27a to be described later to the heart sound acquisition device 20C according to the fifth embodiment. The other configurations of the heart sound acquisition device 20D are the same as those of the heart sound acquisition device 20C according to the fifth embodiment, and thus, the same components are denoted by the same reference numerals as those of the fifth embodiment, and the description of the same components of the heart sound acquisition device 20C will be omitted.

As illustrated in FIG. 9, the heart sound acquisition device 20D includes a hollow disk-shaped pushing control portion 27a extending inward from an end of the cover 27 on a side to be in contact with a human body 5. The pushing control portion 27a has a hole having a size that allows a first sensor 22 to pass through the hole in the pushing control portion 27a in the central part.

The heart sound acquisition device 20D has the pushing control portion 27a, whereby it is possible to further suppress the first sensor 22 from being pushed into the cover 27. As a result, it is possible to further reduce the influence of the strength of the force from the user to press the heart sound acquisition device 20D against the human body 5 of the subject on the pressing pressure of the first sensor 22 against the human body 5. In addition, since the heart sound acquisition device 20D includes the pushing control portion 27a, it is possible to prevent dust from entering the cover 27.

### Seventh Embodiment

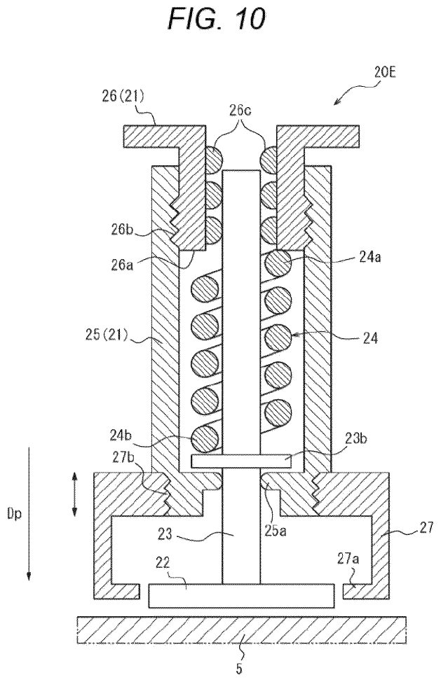

A heart sound acquisition device 20E according to the seventh embodiment will be described with reference to FIG. 10. The heart sound acquisition device 20E is obtained by modifying the heart sound acquisition device 20D according to the sixth embodiment such that the cover 27 is screwed into a spiral groove formed in the cylindrical portion of the first member 25, and the position of the cover 27 in the pressing direction Dₚ can be adjusted by rotating the cover 27. The other configurations of the heart sound acquisition device 20E are the same as those of the heart sound acquisition device 20D according to the sixth embodiment, and thus, the same components are denoted by the same reference numerals as those of the sixth embodiment, and the description of the same components of the heart sound acquisition device 20D will be omitted.

In the heart sound acquisition device 20E, the first member 25 that is integral in the heart sound acquisition device 20D according to the sixth embodiment is separated into the cover 27 and the remaining portion as illustrated in FIG. 10. The remaining portion is referred to as a first member 25 in the following description of the seventh embodiment. The first member 25 of the heart sound acquisition device 20E has a spiral groove on the outer side surface at an end on the second side in the pressing direction.

On the other hand, the cover 27 is rotatably and slidably screwed into the spiral groove formed on the outer periphery of the first member 25 on the first side. As a result, by rotating the cover 27 with respect to the first member 25, the relative position of the cover 27 with respect to the first member 25 changes in the pressing direction Dₚ. When the cover 27 is displaced forward or backward in the pressing direction Dₚ with respect to an annular protrusion 25a of the first member 25, the biasing force on a movable member 23 by a biasing portion 24 at the time of acquisition of the heart sound changes. For example, when the cover 27 is rotated to move the cover 27 in the direction opposite to the pressing direction Dₚ, a first end 24a and a second end 24b of the biasing portion 24 approach each other at the time of pressing the human body 5, so that a larger biasing force is applied to the movable member 23. A threaded portion 27b of the cover 27 screwed with the first member 25 is an adjustment portion that adjusts the biasing force of the biasing portion 24 on the movable member 23.

As described above, according to the heart sound acquisition device 20E according to the seventh embodiment, it is possible to adjust the pressure applied by the first sensor 22 to press the human body by adjusting the position of the cover 27 in the pressing direction Dₚ with respect to the annular protrusion 25a in addition to adjusting the position of the second member 26.

### Eighth Embodiment

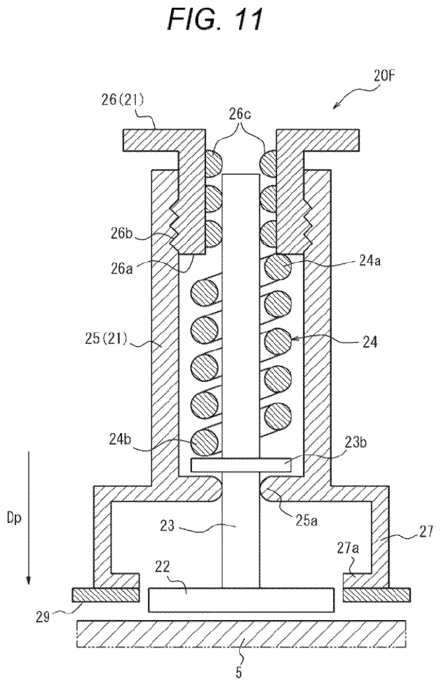

A heart sound acquisition device 20F according to the eighth embodiment will be described with reference to FIG. 11. The heart sound acquisition device 20F is obtained by adding an additional component 29 on the surface of the pushing control portion 27a of the cover 27 to be in contact with the human body 5 in the heart sound acquisition device 20D according to the sixth embodiment. The other configurations of the heart sound acquisition device 20E are the same as those of the heart sound acquisition device 20D according to the sixth embodiment, and thus, the same components are denoted by the same reference numerals as those of the sixth embodiment, and the description of the same components of the heart sound acquisition device 20D will be omitted as appropriate.

The additional component 29 can be, for example, a second sensor that acquires biological information other than heart sounds. The second sensor can include, for example, an electrode of an electrocardiograph. By using the heart sound acquisition device 20F, it is possible to simultaneously or sequentially acquire heart sounds by the first sensor 22 and acquire the biological information by the second sensor using the same device.

Further, the additional component 29 may include an adhesive member that can be peeled off from the human body 5 in order to affix the cover 27 to the human body 5. In this case, the user can fix the pushing control portion 27a of the cover 27 to the human body 5 to continuously acquire the heart sound from the same place of the human body 5. Further, the additional component 29 may include both the second sensor and the adhesive member, which enables both measurement by the second sensor and fixation of the heart sound acquisition device 20F to the heart sound acquisition site of the human body 5.

### Ninth Embodiment

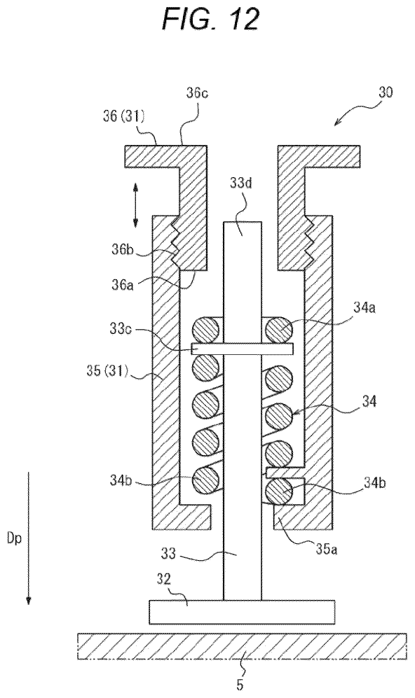

In the first to eighth embodiments, the biasing portions 14 and 24 exert a force in a direction of increasing the distance between the first ends 14a and 24a and the second ends 14b and 24b. On the other hand, a heart sound acquisition device 30 according to the ninth embodiment illustrated in FIG. 12 biases a movable member 33 using a force for reducing the distance between a first end 34a and a second end 34b of a biasing portion 34. Portions of the heart sound acquisition device 30 according to the ninth embodiment different from the heart sound acquisition device 20 according to the second embodiment will be described below, as appropriate.

The heart sound acquisition device 30 includes a main body 31, the movable member 33 having a first sensor 32, and the biasing portion 34. The main body 31 includes a first member 35 and a second member 36. The main body 31 (first member 35, second member 36), the first sensor 32, the movable member 33, and the biasing portion 34 correspond to the main body 21 (first member 25, second member 26), the first sensor 22, the movable member 23, and the biasing portion 24 in the second embodiment, respectively.

The biasing portion 34 can be, for example, a spring in a state of being extended from a natural length. The biasing portion 34 generates a force in a direction in which the first end 34a approaches the second end 34b. The first end 34a of the biasing portion 34 is engaged with a protrusion 33c disposed on the movable member 33 on the first side. The second end 34b of the biasing portion 34 is engaged with the first member 35 on the second side. Thus, the biasing portion 34 biases the movable member 33 in the pressing direction Dₚ with respect to the first member 35. Therefore, the first sensor 32 attached to the movable member 33 can be pressed against the human body 5.

In the heart sound acquisition device 30, a surface 36a of the second member 36 on the second side is a first movement restriction portion that restricts the movement of the movable member 33 in the direction opposite to the pressing direction Dₚ. An annular protrusion 35a of the first member 35 with which the biasing portion 34 is engaged is a second movement restriction portion that restricts the movement of the movable member 33 and the biasing portion 34 in the pressing direction Dₚ.

In addition, the user may adjust the pressure for pressing the heart sound acquisition device 30 in the pressing direction Dₚ on the basis of the positional relationship between the movable member 33 and the second member 36. For example, the user may position the movable member 33 with respect to the main body 31 by aligning a mark M disposed on an upper part 33d of the movable member 33 with an upper surface 36c of a flange of the second member 36.

By rotating the second member 36 to displace the second member 36 forward or backward in the pressing direction Dₚ, the pressing force applied to the movable member 33 by the biasing portion 34 changes when the mark M is aligned with the upper surface of the flange. Therefore, a threaded portion 36b of the second member 36 screwed with the first member 35 functions as an adjustment portion that adjusts the biasing force of the biasing portion 34 on the movable member 33.

### Tenth Embodiment

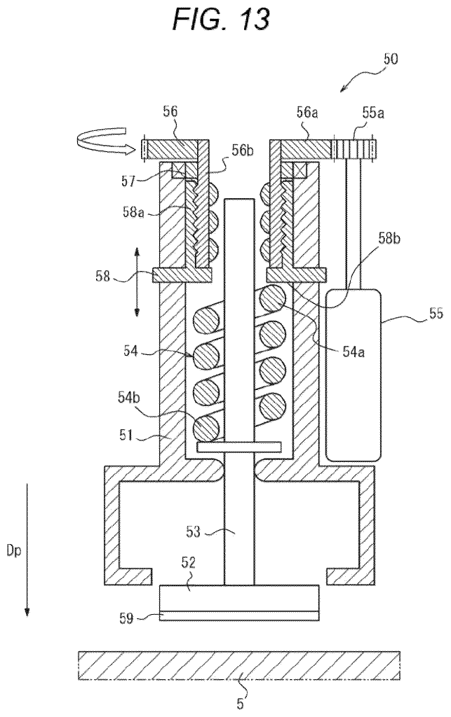

A heart sound acquisition device 50 according to the tenth embodiment will be described with reference to FIG. 13. The heart sound acquisition device 50 includes a main body 51, a movable member 53 having a first sensor 52, a biasing portion 54, and a motor 55. The main body 51 includes a rotary member 56, a bearing 57, and a vertical member 58. The heart sound acquisition device 50 may further include a third sensor 59.

The main body 51, the first sensor 52, the movable member 53, and the biasing portion 54 have configurations same as or similar to the configurations of the first member 25, the first sensor 22, the movable member 23, and the biasing portion 24 in the heart sound acquisition device 20D according to the sixth embodiment, respectively.

The motor 55 generates power to rotate around a rotation shaft. The motor 55 includes a first gear 55a rotatable about the rotation shaft. The motor 55 is configured to be able to control the rotation amount of the first gear 55a. The rotary member 56 includes a second gear 56a meshing with the first gear 55a of the motor 55, and a cylindrical portion 56b extending from the second gear 56a toward the second side. The cylindrical portion 56b extends in the main body 51 to the first side. A spiral groove is formed on an outer surface of the cylindrical portion 56b. The cylindrical portion 56b is configured to be rotatable inside the cylindrical portion on the first side of the main body 51 via the bearing 57.

The vertical member 58 is a cylindrical member that is in contact with the inner surface of the main body 51 and is slidable forward and backward in the pressing direction Dₚ. For example, a part of the vertical member 58 is fitted into a hole or a groove formed in the main body 51 and extending in the pressing direction Dₚ, so that the movement in a direction other than the pressing direction Dₚ (vertical direction in FIG. 13) is restricted. A spiral groove rotatably and slidably screwed with a spiral groove formed on the outer surface of the rotary member 56 is formed on the inner surface of the vertical member 58.

The third sensor 59 is a pressure sensor that measures the pressure applied by the first sensor 52 to press the human body 5. The third sensor 59 is disposed, for example, on the first sensor 52 or adjacent to the first sensor 52. The arrangement of the third sensor 59 is not limited to the third sensor 59 disposed on the first sensor 52 or adjacent to the first sensor 52, and for example, the third sensor can be placed on the movable member 53. In addition, in a case where the first sensor 52 is a sensor of a type that detects sound by pressure, the third sensor 59 is unnecessary. The third sensor 59 may be any of various sensors such as a strain gauge pressure sensor, a metal gauge pressure sensor, a semiconductor pressure sensor (piezoresistive pressure sensor), a capacitance pressure sensor, a vibration pressure sensor, and an optical fiber pressure sensor.

With the above configuration, in the heart sound acquisition device 50, when the motor 55 is driven to rotate the first gear 55a, the second gear 56a meshing with the first gear rotates, so that the entire rotary member 56 rotates. As the cylindrical portion 56b of the rotary member 56 rotates, the vertical member 58 screwed into the cylindrical portion 56b moves in either the upward direction or the downward direction. Therefore, the position of a lower surface 58b of the vertical member 58 can be changed by controlling the motor 55.

The lower surface 58b of the vertical member 58 is in contact with a first end 54a of the biasing portion 54. The lower surface 58b of the vertical member 58 functions as a first movement restriction portion. The biasing force of the biasing portion 54 on the movable member 53 is changed by moving the position of the lower surface 58b of the vertical member 58 forward or backward in the pressing direction Dₚ. Therefore, the vertical member 58 functions as the adjustment portion. The motor 55, the rotary member 56, the bearing 57, and the vertical member 58 function as a pressure adjustment mechanism that adjusts the pressure applied by the first sensor 52 to press the human body 5.

In the present disclosure, the pressure adjustment mechanism refers to a mechanism configured to change the biasing force on the movable member by system control and adjusting the pressure applied by the heart sound sensor to press the human body 5. The pressure adjustment mechanism is one mode of the adjustment portion.

According to the heart sound acquisition device 50 according to the tenth embodiment, it is possible to control the motor 55 to adjust the pressure applied by the first sensor 52 to press the human body 5. Note that the method for driving the vertical member 58 is not limited to the above-described method. The heart sound acquisition device 50 may include a drive unit other than the motor 55.

In addition, in a case where the heart sound acquisition device 50 includes the third sensor 59, the heart sound acquisition device 50 can adjust the pressure applied by the first sensor 52 to press the human body 5 by the pressure adjustment mechanism while measuring the pressure.

### Eleventh Embodiment

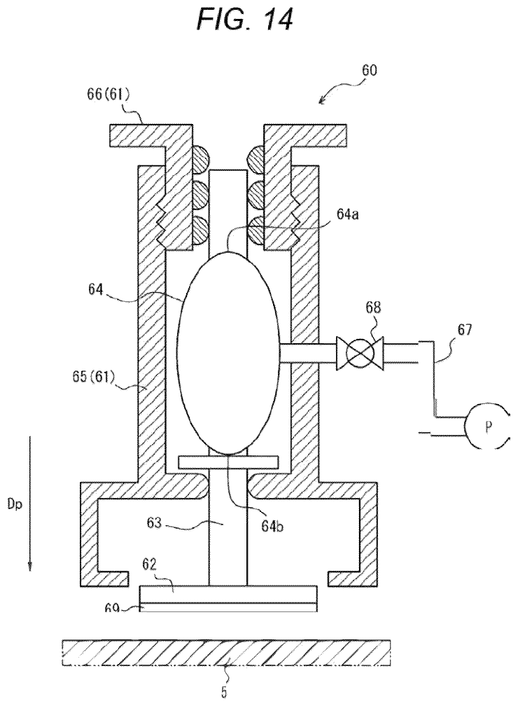

A heart sound acquisition device 60 according to the eleventh embodiment will be described with reference to FIG. 14. The heart sound acquisition device 60 includes a main body 61, a movable member 63 having a first sensor 62, and a biasing portion 64. The heart sound acquisition device 60 may further include a third sensor 69. The main body 61 includes a first member 65 and a second member 66. The configuration of the main body 61 is the same as or similar to the main body 21 of the heart sound acquisition device 20D according to the sixth embodiment except that the first member 65 includes a hole through which an air tube 67 passes, and thus the description the main body 61 will be omitted.

The first sensor 62 that acquires the heart sound may be configured in the same manner as the first sensor 22 in the sixth embodiment. Similar to the heart sound acquisition device 50 according to the tenth embodiment, the third sensor 69 may be disposed on the first sensor 62 or adjacent to the first sensor 62 in order to measure the pressure applied by the first sensor 62 to press the human body 5. The third sensor 69 is similar to the third sensor 59 in the tenth embodiment.

The movable member 63 may be configured integrally with the biasing portion 64. For example, the movable member 63 may be divided into two sections along the pressing direction Dₚ, and the biasing portion 64 may be disposed between the two sections. In the example illustrated in FIG. 14, the biasing portion 64 can be a balloon having elasticity and configured to expand and contract by introducing air into the inside and discharging air from the inside. In this case, the heart sound acquisition device 60 includes an air tube 67 communicating with the inside of the biasing portion 64, a control valve 68 positioned in the middle of the air tube 67 and configured to control opening and closing, and a pump P that sends air to the air tube 67 or discharges air from the air tube 67.

With the above configuration, the heart sound acquisition device 60 can send or discharge air to or from the inside of the balloon serving as the biasing portion 64 to adjust an amount of air in the balloon under the control of the pump P and the control valve 68, whereby the biasing force of the biasing portion 64 on the movable member 63 can be adjusted. That is, the pump P, the air tube 67, and the control valve 68 function as a pressure adjustment mechanism that adjusts the pressure applied by the first sensor 62 to press the heart sound acquisition site of the human body 5.

In addition, in a case where the heart sound acquisition device 60 includes the third sensor 69, the heart sound acquisition device 60 can adjust the pressure applied by the first sensor 62 to press the human body 5 by the pressure adjustment mechanism while measuring the pressure.

### Twelfth Embodiment

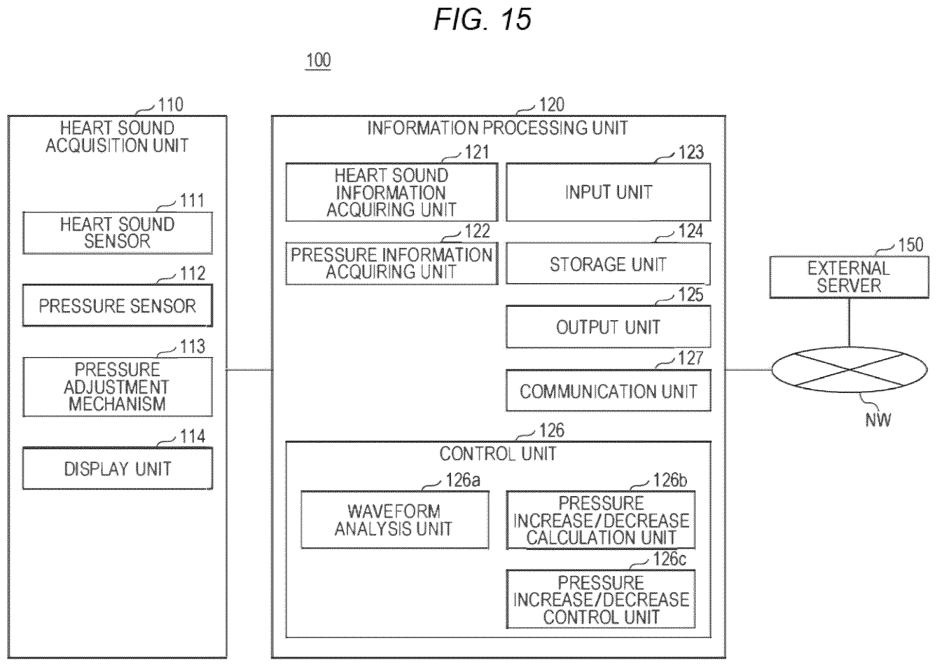

The heart sound acquisition device 50 according to the tenth embodiment and the heart sound acquisition device 60 according to the eleventh embodiment can control the pressure for pressing the heart sound acquisition site of the human body 5 using an information processing system at the time of measuring the heart sound. The twelfth embodiment will describe a heart sound acquisition system 100 using the heart sound acquisition devices 50 and 60 configured to control the pressure as a heart sound acquisition unit 110 with reference to FIG. 15.

As illustrated in FIG. 15, the heart sound acquisition system 100 includes a heart sound acquisition unit 110 and an information processing unit 120. The heart sound acquisition unit 110 and the information processing unit 120 may be individual devices mounted in different hardware. In this case, the heart sound acquisition unit 110 and the information processing unit 120 are connected by wired or wireless communication means. In another embodiment, the heart sound acquisition unit 110 and the information processing unit 120 may be a single device mounted on one piece of hardware.

The heart sound acquisition unit 110 can be, for example, the heart sound acquisition device 50 according to the tenth embodiment and the heart sound acquisition device 60 according to the eleventh embodiment. The heart sound acquisition unit 110 can include, for example, a heart sound sensor 111, a pressure sensor 112, and a pressure adjustment mechanism 113. The heart sound acquisition unit 110 may further include a display unit 114.

The heart sound sensor 111 is a first sensor that contacts a human body to acquire a heart sound. The heart sound sensor 111 corresponds to the first sensor 52 of the heart sound acquisition device 50 according to the tenth embodiment and the first sensor 62 of the heart sound acquisition device 60 according to the eleventh embodiment.

The pressure sensor 112 is a third sensor that measures the pressure applied by the heart sound sensor 111 to press the human body. The pressure sensor 112 corresponds to the third sensor 59 of the heart sound acquisition device 50 according to the tenth embodiment and the third sensor 69 of the heart sound acquisition device 60 according to the eleventh embodiment. The pressure sensor 112 is not always necessary.

The pressure adjustment mechanism 113 adjusts the pressure applied by the heart sound sensor 111 to press a heart sound acquisition site of the human body 5. The pressure adjustment mechanism 113 includes the motor 55 of the heart sound acquisition device 50 according to the tenth embodiment, and the pump P and the control valve 68 of the heart sound acquisition device 60 according the eleventh embodiment. The pressure adjustment mechanism 113 may include a means using an electromagnetic force in addition to a mechanical means using a motor, a gear, or the like, and a means using a gas such as air. The means using an electromagnetic force may include, for example, a means that controls the position of a member in the heart sound acquisition unit 110 using a magnetic body and a coil to thereby adjust the pressure applied by the heart sound sensor 111 to press the human body.

The display unit 114 can include a light emitting diode (LED) and/or a liquid crystal display. The display unit 114 may display the acquisition state of the heart sound including an error or the like under the control of the information processing unit 120. The display unit 114 may be configured to output sound, light, vibration, or the like other than an image. The display unit 114 is not always necessary.

The information processing unit 120 executes various processes on the basis of a signal of the heart sound acquired by the heart sound acquisition unit 110. The information processing unit 120 controls the pressure adjustment mechanism 113 of the heart sound acquisition unit 110. The information processing unit 120 may be a dedicated computer used for the heart sound acquisition system 100 or a general-purpose computer. When being a general-purpose computer, the information processing unit 120 may be, for example, any of a tablet terminal, a smartphone, a notebook personal computer (PC), a desktop PC, a workstation, and the like. The functions of the information processing unit 120 may be distributed and arranged in a plurality of devices instead of a single device.

The information processing unit 120 can include a heart sound information acquiring unit 121, an input unit 123, a storage unit 124, an output unit 125, and a control unit 126. The information processing unit 120 may further include at least one of a pressure information acquiring unit 122 or a communication unit 127. The pressure information acquiring unit 122 and the communication unit 127 are not always necessary.

The heart sound information acquiring unit 121 acquires a heart sound signal acquired by the heart sound sensor 111 from the heart sound acquisition unit 110. The pressure information acquiring unit 122 acquires, from the heart sound acquisition unit 110, information regarding the pressure applied by the heart sound sensor 111 to press the human body, the pressure being acquired by the pressure sensor 112. The heart sound information acquiring unit 121 and the pressure information acquiring unit 122 can transmit and receive information to and from the heart sound acquisition unit 110 by wireless or wired communication means.

The input unit 123 can include one or more input interfaces that detect a user's input and acquire input information based on the user's operation. For example, the input unit 123 can be a physical key, a capacitance key, a touch screen integrated with the display device of the output unit 125, a microphone that receives voice input, or the like, but is not limited to the input units described above. The input unit 123 can be used by the user to, for example, input or change the condition of the pressure applied by the heart sound sensor 111 to press the human body at the time of measurement using the heart sound acquisition unit 110.

The storage unit 124 is, for example, a semiconductor memory, a magnetic memory, an optical memory, or the like, but is not limited to the examples described above. The storage unit 124 may function as, for example, a main storage device, an auxiliary storage device, or a cache memory. The storage unit 124 stores any information used for the operation of the heart sound acquisition unit 110. For example, the storage unit 124 may sequentially store a system program, an application program, information acquired by the heart sound information acquiring unit 121 and the pressure information acquiring unit 122, and the like. When the heart sound is acquired by the heart sound acquisition unit 110, the storage unit 124 may store a pressure value of the pressure applied by the heart sound sensor 111 to press the heart sound acquisition site of the human body 5. A part of the storage unit 124 may be installed outside the information processing unit 120. In that case, a part of the storage unit 124 installed outside may be connected to the information processing unit 120 via any interface.

The output unit 125 can include one or more output interfaces that output information and notify the user of the output information. For example, the output unit 125 can include a display device that outputs information as an image and/or a speaker that outputs information by voice, but is not limited to the examples described above. The output unit 125 may be configured to output information in various modes. The output unit 125 can display a measurement error, a cause of the measurement error, a guidance on how to deal with the error, and the like under the control of the control unit 126.

The control unit 126 can include at least one processor, at least one dedicated circuit, or a combination of at least one processor and at least one dedicated circuit. The processor can be a general-purpose processor such as a central processing unit (CPU) or a graphics processing unit (GPU), or a dedicated processor specialized for specific processing. The dedicated circuit can be, for example, a field-programmable gate array (FPGA) or an application specific integrated circuit (ASIC).

The control unit 126 executes processing related to the operation of the information processing unit 120 while controlling the respective units of the information processing unit 120. As illustrated in FIG. 15, the control unit 126 includes functional blocks that are a waveform analysis unit 126a, a pressure increase/decrease calculation unit 126b, and a pressure increase/decrease control unit 126c. Each of the functional blocks may be a hardware module or a software module. The processing executed by each of the functional blocks can be rephrased as processing executed by the control unit 126.

The waveform analysis unit 126a is configured to analyze the waveform (heart sound waveform) of the heart sound signal acquired by the heart sound information acquiring unit 121. The waveform analysis unit 126a can detect, for example, the amplitude of the heart sound signal.

The pressure increase/decrease calculation unit 126b is configured to determine an increase or decrease in pressure applied by the heart sound sensor 111 to press the human body at the time of next acquisition of the heart sound according to the amplitude of the heart sound signal detected by the waveform analysis unit 126a.

The pressure increase/decrease control unit 126c is configured to transmit a control signal to the pressure adjustment mechanism 113 of the heart sound acquisition unit 110 according to an increase or decrease in the pressure determined by the pressure increase/decrease calculation unit 126b to adjust the pressure that is applied by the heart sound sensor 111 of the heart sound acquisition unit 110 to press the human body.

The control unit 126 may have a function of acquiring a heart sound signal and performing various analyses after adjusting the pressure applied by the heart sound sensor 111 of the heart sound acquisition unit 110 to press the human body. For example, the control unit 126 may calculate a diagnostic index related to the subject by analyzing the heart sound signal. Furthermore, for example, the control unit 126 may perform screening for diseases related to cardiac function such as valvular disease and heart failure, and/or respiratory diseases such as sleep apnea syndrome.

The communication unit 127 can transmit and receive information to and from the external server 150 via a network NW. The network NW can be, for example, the Internet, a dedicated communication line, or the like. The external server 150 can be, for example, a server of a hospital used by the subject who performs measurement at home, or a dedicated server of the heart sound acquisition system 100 installed in a cloud. The communication unit 127 includes at least one communication interface. Examples of the communication interface include a local area network (LAN) interface, a Bluetooth® interface, and an interface of a mobile communication system such as 4th generation (4G) and 5th generation (5G).

The control unit 126 may transmit heart sound data based on the heart sound signal of the subject and a diagnostic index obtained as a result of analyzing the heart sound data to the external server 150 via the communication unit 127. The information stored in the external server 150 can be used as information for assisting a medical worker such as a doctor to confirm the state of a patient and intervene in a prescription change or the like, as necessary.

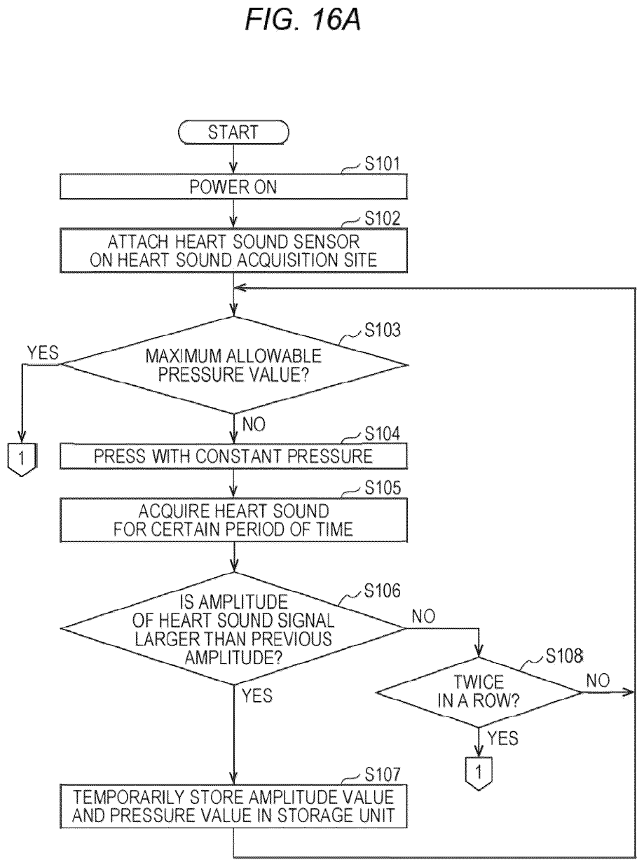

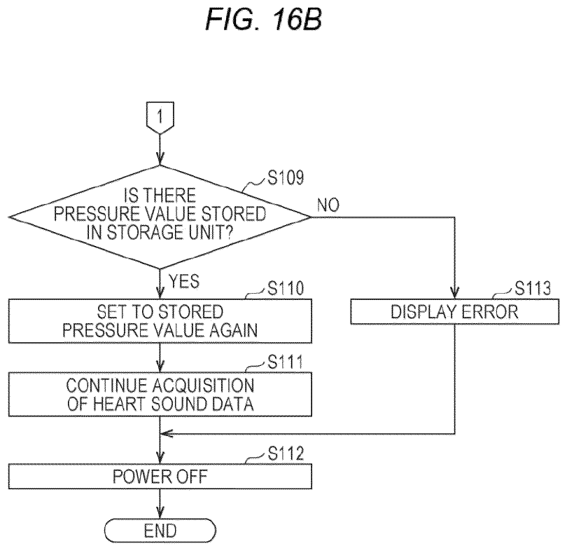

A method for acquiring a heart sound using the heart sound acquisition system 100 according to the twelfth embodiment will be described below with reference to FIGS. 16A and 16B.

First, the user turns on the heart sound acquisition system 100 and activates the heart sound acquisition system 100 (step S101).

The user attaches the heart sound sensor 111 of the heart sound acquisition unit 110 on the heart sound acquisition site of the subject (step S102). The heart sound acquisition site is a predetermined position of the left chest of the subject. The heart sound acquisition unit 110 is positioned at a suitable location for acquiring the heart sound.

After the heart sound acquisition unit 110 is attached on the heart sound acquisition site, the control unit 126 determines whether or not the pressure applied by the heart sound sensor 111 to press the human body exceeds a predetermined maximum allowable pressure value (step S103). The maximum allowable pressure value can be set to such a value that, when the pressure value at the time of measuring the heart sound exceeds this value, the pressure is considered to be too high and inappropriate. The pressure value is acquired from the pressure sensor 112 of the heart sound acquisition unit 110 via the pressure information acquiring unit 122. The pressure value is set to an initial value smaller than the maximum allowable pressure value at an initial stage, and thus, the determination in step S103 is No.

The control unit 126 controls the pressure adjustment mechanism 113 so that the heart sound acquisition unit 110 presses with a constant pressure (step S104). The constant pressure can be, for example, 0.5 N (0.5 newtons).

The control unit 126 acquires a signal of the heart sound of the subject detected by the heart sound sensor 111 of the heart sound acquisition unit 110 via the heart sound information acquiring unit 121 for a certain period of time (step S105). The certain period of time can be, for example, three seconds.

The control unit 126 detects the magnitude of the amplitude from the acquired heart sound signal. When the magnitude of the amplitude of the acquired heart sound signal is larger than the amplitude of the previously acquired heart sound signal (Yes in step S106), the control unit 126 stores the detected amplitude value and the pressure value in the storage unit 124 (step S107). When passing step S106 for the first time, the control unit 126 always proceeds to step S107.

After step S107, the processing of the control unit 126 returns to step S103. When the pressure value obtained from the pressure sensor 112 exceeds the maximum allowable pressure value in step S103 (Yes in step S103) after the processing from steps S104 to S107 is repeated a plurality of times, the control unit 126 proceeds to step S109.

When the amplitude of the heart sound signal is not larger than the amplitude of the previously acquired heart sound signal in step S106 (No in step S106), the control unit 126 proceeds to step S108. When it is determined in step S106 that the amplitude of the heart sound signal is not larger than the previously acquired amplitude twice in a row (Yes in step S108) before the processing proceeds to step S108, the processing of the control unit 126 proceeds to step S109. On the other hand, when the determination that the amplitude of the heart sound signal is not larger than the previously acquired amplitude (No in step S106) is not performed twice in a row in step S106 (No in step S108), the control unit 126 returns to step S103.

Generally, when the heart sound sensor 111 gradually increases the value of the pressure applied by the heart sound sensor 111 to press the heart sound acquisition site of the human body 5 from a low value, the amplitude of the heart sound signal gradually increases and peaks at an optimum pressure value. When the pressure value is further increased from the value at which the peak value of the amplitude is obtained, the amplitude of the heart sound signal gradually decreases. Therefore, when the amplitude of the heart sound signal falls below the previous amplitude in step S106 (No in step S106), it is likely that the amplitude is the maximum at the previous acquisition of the heart sound signal. Further, by confirming in step S108 that the state in which the amplitude of the heart sound signal is not larger than the previous amplitude continues twice in a row (Yes in step S108), it is possible to reliably acquire the peak value of the heart sound signal.

In step S109, the control unit 126 checks whether or not there is a pressure value stored in the memory of the storage unit 124 (step S109). When the pressure value is stored in the storage unit 124 (Yes in step S109), the stored pressure value is the pressure value that has reached the maximum value and stored in step S107. In this case, the control unit 126 sets the pressure for pressing the heart sound acquisition site again to the pressure value stored in the storage unit 124 (step S110).

The control unit 126 continuously acquires heart sound data for a predetermined time in a state where the amplitude of the heart sound signal is maximum (step S111). As a result, the heart sound acquisition system 100 can acquire the heart sound with the optimum pressure value. The control unit 126 may transmit heart sound data and/or a diagnostic index obtained by the analysis of the heart sound data to the external server 150 during or after the acquisition of the heart sound.

On the other hand, when there is no pressure value stored in the memory in step S109 (No in step S109), it means that the amplitude of the heart sound signal has never increased even though the pressure value of the heart sound sensor 111 has been increased. In that case, the control unit 126 displays an error on the display unit 114 and/or the output unit 125 of the heart sound acquisition unit 110 (step S113).

After step S111 or step S113, the power of the heart sound acquisition system 100 is turned off manually by the user or by a program of the system, and the processing ends (step S112).

As described above, the heart sound acquisition system 100 according to the twelfth embodiment detects a pressure at which the amplitude of the heart sound signal is maximized while automatically changing the pressure applied by the heart sound acquisition unit 110 to press the human body under the control of the control unit 126. Thus, the heart sound acquisition system 100 can acquire the heart sound with high reproducibility with the optimum pressure even if the user does not have medical expertise.

In the heart sound acquisition system 100 according to the twelfth embodiment, the pressure sensor 112 is disposed in the heart sound acquisition unit 110, and the control unit 126 controls the pressure of the heart sound sensor 111 on the basis of the pressure information obtained from the pressure sensor 112. However, the heart sound acquisition system 100 can be configured not to include the pressure sensor 112. When a configuration including a motor that can control the rotation amount in the forward direction and the reverse direction is used as the pressure adjustment mechanism 113 of the heart sound acquisition unit 110, for example, it is possible to associate, instead of the pressure information acquired from the pressure sensor 112, the rotation amount of the motor from the initial state with the amplitude of the heart sound signal.

In this case, the maximum allowable pressure value in step S103 corresponds to the maximum allowable motor rotation amount. Further, pressing with the constant pressure in step S104 is rotation of a constant rotation amount by the motor. In step S107, the storage unit 124 sequentially stores the amplitude value and the rotation amount of the motor. In this manner, the rotation amount of the motor at which the maximum amplitude is obtained can be set in step S110. As described above, the control unit 126 controls the pressure adjustment mechanism 113 to adjust the pressure applied by the heart sound sensor 111 to press the heart sound acquisition site of the subject to the pressure at which the amplitude of the heart sound signal is maximized without the pressure sensor 112.

### Thirteenth Embodiment

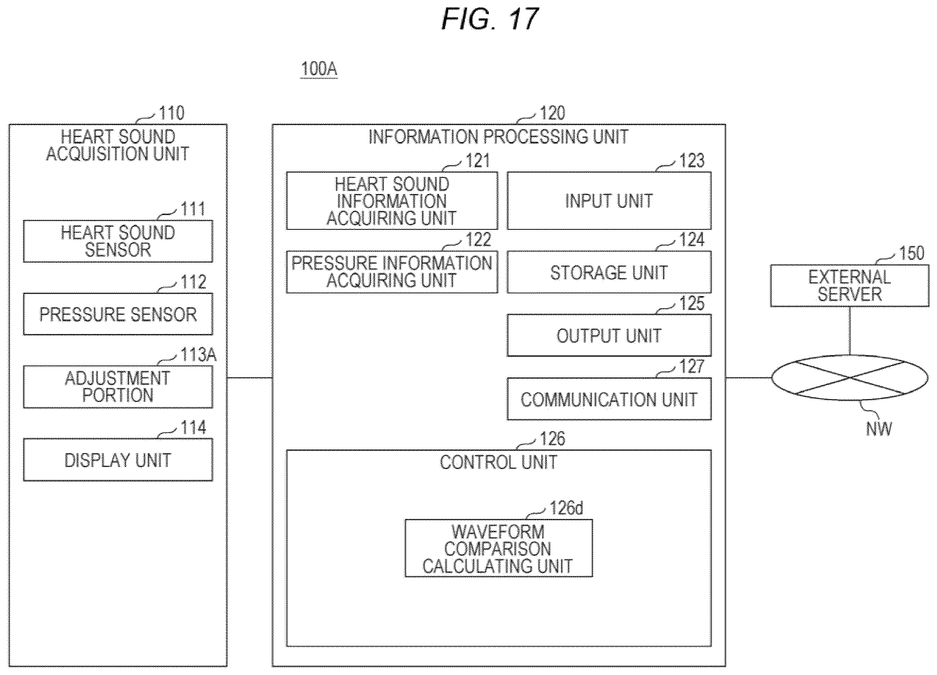

A heart sound acquisition system 100A according to the thirteenth embodiment of the present disclosure will be described with reference to FIG. 17. This heart sound acquisition system 100A stores in advance a heart sound waveform acquired remotely or acquired by a medical worker such as a doctor at a medical institution such as a hospital as a reference waveform. When the user acquires a heart sound, the heart sound acquisition system 100A checks that an appropriate waveform has been acquired as compared with the stored reference waveform. When an appropriate heart sound waveform cannot be acquired, the heart sound acquisition system 100A displays an error and/or guidance on how to deal with the error.

The heart sound acquisition system 100A is similar to the heart sound acquisition system 100 according to the twelfth embodiment, and thus, the same or similar components are denoted by the same reference numerals as those of the heart sound acquisition system 100. Differences from the heart sound acquisition system 100 will be described below.

The heart sound acquisition unit 110 can be a device that manually adjusts pressure like the heart sound acquisition devices 10, 20, 20A to 20F, and 30 according to the first to ninth embodiments, or a device having a controllable pressure adjustment mechanism 113 like the heart sound acquisition device 50 according to the tenth embodiment and the heart sound acquisition device 60 according to the eleventh embodiment. In the former device, each of the first sensors 12, 22, and 32 serves as the heart sound sensor 111. In addition to the heart sound sensor 111, a pressure sensor 112 can be mounted. Note that the pressure sensor 112 is not always necessary.

An adjustment portion 113A adjusts the pressure applied by the heart sound sensor 111 to press the heart sound acquisition site of the human body 5. The adjustment portion 113A may be configured to manually adjust the pressure like the adjustment portions of the heart sound acquisition devices according to the first to ninth embodiments. The adjustment portion 113A may be a pressure adjustment mechanism that automatically adjusts the pressing force under the control of the information processing unit 120.

An input unit 123 of the heart sound acquisition system 100A can receive input of data of a reference waveform when a medical worker such as a doctor acquires a heart sound. A storage unit 124 can store data of the reference waveform of the heart sound of the subject.

A control unit 126 includes a waveform comparison calculating unit 126d instead of the waveform analysis unit 126a, the pressure increase/decrease calculation unit 126b, and the pressure increase/decrease control unit 126c in the twelfth embodiment. The waveform comparison calculating unit 126d is configured to compare the heart sound waveform acquired by the heart sound sensor 111 via the heart sound information acquiring unit 121 with the reference waveform stored in the storage unit 124. The heart sound waveform acquired by the user with the heart sound sensor 111 is referred to as a measurement waveform. The measurement waveform of the heart sound is included in the data indicating the heart sound. The waveform comparison calculating unit 126d determines the similarity between the measurement waveform and the reference waveform. Dynamic time warping (DTW), for example, can be used as a method for determining the similarity between the measurement waveform and the reference waveform. The DTW is a method used to measure similarity between time-series data. The waveform comparison calculating unit 126d can instruct to acquire a heart sound or display an error and guidance using the output unit 125 according to the determination result of the similarity between the measurement waveform and the reference waveform.

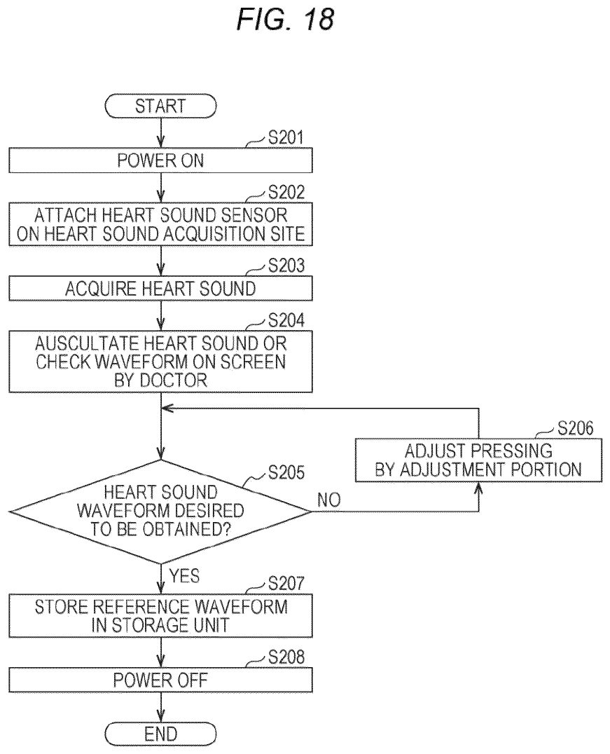

Next, a procedure of storing a reference waveform performed by a medical worker such as a doctor prior to measurement using the heart sound acquisition system 100A will be described with reference to FIG. 18. This procedure may be performed by a medical worker in a hospital or the like. In addition, this procedure can be performed by a subject at home in accordance with an instruction remotely given from a doctor. When a pressure adjustment mechanism that can be controlled by the information processing unit 120 is used as the adjustment portion 113A, this procedure may be performed using a system different from the heart sound acquisition system 100A used by the user at home. In the following, an example of using the same system as the heart sound acquisition system 100A used at home will be described.

First, the power of the heart sound acquisition system 100A is turned on by the medical worker or the user at home (step S201).

Next, the medical worker or the user attaches the heart sound sensor 111 of the heart sound acquisition unit 110 on the heart sound acquisition site of the subject (step S202). The information processing unit 120 can detect that the heart sound acquisition unit 110 is attached on the subject based on a signal received by the heart sound information acquiring unit 121 and/or the pressure information acquiring unit 122.

The heart sound acquisition unit 110 acquires the heart sound from the heart sound sensor 111 (step S203). The heart sound signal acquired by the heart sound sensor 111 is transmitted to the control unit 126 of the information processing unit 120 via the heart sound information acquiring unit 121.

The control unit 126 outputs the heart sound signal as a sound and/or a waveform from the output unit 125. The heart sound acquisition unit 110 may be able to directly output the heart sound without the information processing unit 120 so that the medical worker can directly auscultate the heart sound. The medical worker such as a doctor auscultates the heart sound and/or checks the heart sound waveform with the output unit 125 (step S204).

The medical worker such as a doctor determines whether or not a heart sound desired to be obtained is output (step S205). When a pressure adjustment mechanism that can be controlled by the information processing unit 120 is used as the adjustment portion 113A, the control unit 126 may wait for determination as to whether or not a heart sound waveform desired to be obtained from the medical worker such as a doctor is output via the input unit 123. The medical worker such as a doctor may input into the input unit 123, whether or not the heart sound waveform desired to be obtained has been obtained after auscultating the heart sound and/or visually checking the heart sound waveform.

When it is determined in step S205 that the desired heart sound waveform cannot be obtained from the medical worker (No in step S205), the pressure applied by the heart sound sensor 111 to press the heart sound acquisition site is adjusted using the adjustment portion 113A (step S206). This adjustment may be performed by the medical worker manually by adjusting the adjustment portion 113A. When the heart sound acquisition unit 110 has a pressure adjustment mechanism that can be controlled by the information processing unit 120 as the adjustment portion 113A, this adjustment can be performed by the medical worker operating the input unit 123. After step S206, the procedure of the flowchart of FIG. 18 proceeds to step S205 again.

When it is determined in step S205 that the desired heart sound waveform has been obtained from the medical worker (Yes in step S205), the medical worker operates the input unit 123, so that the control unit 126 stores the acquired heart sound waveform in the storage unit 124 as a reference waveform (step S207). The control unit 126 may acquire a reference pressure, which is a pressure detected by the pressure sensor 112 when the reference waveform is acquired, together with the reference waveform via the pressure information acquiring unit 122, and store the reference pressure in the storage unit 124. It is not always necessary to obtain a reference pressure.

After step S207, the power of the heart sound acquisition system 100A is turned off by manual operation performed by the medical worker or by a program of the system, and the processing ends (step S208).

Note that, in a case where the heart sound acquisition system 100A used by the user is different from the heart sound acquisition system 100A used by the medical worker to acquire the reference waveform, information such as the reference waveform and the reference pressure may be transmitted from the system on the medical worker side to the heart sound acquisition system 100A of the user via a wired or wireless communication means. The heart sound acquisition system 100A on the user side may store the received information such as the reference waveform and the reference pressure in the storage unit 124. It is not always necessary to store the reference pressure in the storage unit 124.

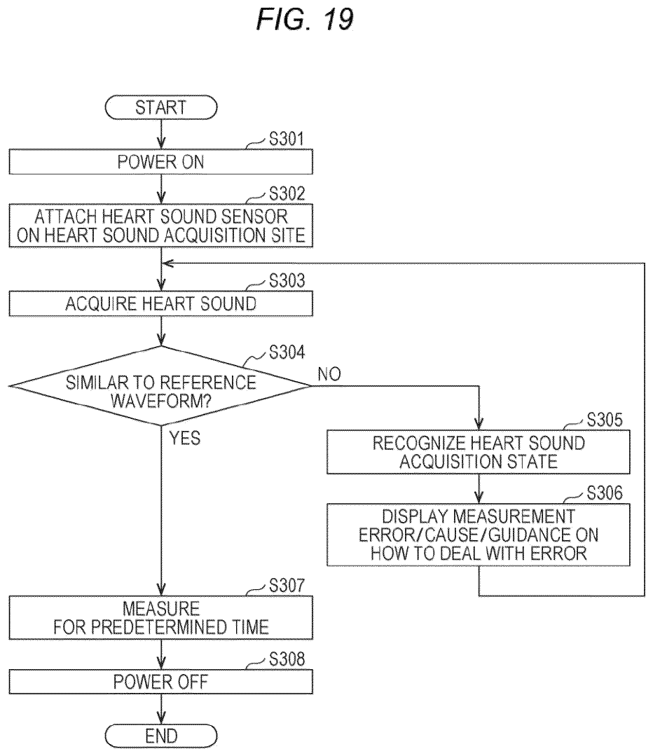

Next, a procedure for acquiring a heart sound performed by the user at home or the like will be described with reference to a flowchart of FIG. 19. The heart sound acquisition unit 110 may initially set the pressure for pressing the heart sound sensor 111 against the heart sound acquisition site at the time of acquiring the heart sound to be the pressure at the time of acquiring the reference waveform.

First, the power of the heart sound acquisition system 100A is turned on by the user (step S301). Thus, each unit of the heart sound acquisition system 100A is activated.

Next, the user attaches the heart sound sensor 111 on the heart sound acquisition site of the subject (step S302). The information processing unit 120 can detect that the heart sound acquisition unit 110 is attached on the subject based on the signal from the heart sound information acquiring unit 121 and/or the pressure information acquiring unit 122.

The heart sound acquisition unit 110 acquires the heart sound by the heart sound sensor 111 in a state where the heart sound sensor 111 is attached on the heart sound acquisition site of the subject (step S303). The heart sound signal acquired by the heart sound sensor 111 is transmitted to the information processing unit 120 via the heart sound information acquiring unit 121. As a result, the information processing unit 120 acquires the heart sound signal.

The control unit 126 of the information processing unit 120 compares the measurement waveform that is the waveform of the acquired heart sound signal with the reference waveform stored in the storage unit 124, and determines the similarity (step S304). The control unit 126 may set a threshold for the similarity, and determine whether they are similar or not by comparing the similarity calculated by a predetermined method such as DTW with the threshold.

When determining that the acquired measurement waveform is not similar to the reference waveform (No in step S304), the control unit 126 recognizes a heart sound acquisition state (step S305). The heart sound acquisition state includes information indicating that the amplitude of the measurement waveform is smaller or larger than the reference waveform by a predetermined percentage or more.

The control unit 126 displays an occurrence of a measurement error, a cause of the error, and/or a guidance on how to deal with the error on the output unit 125 and/or the display unit 114 of the heart sound acquisition unit 110 on the basis of the heart sound acquisition state recognized in step S305 (step S306). The displayed information such as the cause of the error and the way to deal with the error may be stored in advance in the storage unit 124 in association with the assumed heart sound acquisition state. For example, the cause of the error may include insufficient pressure, positional misalignment, noise, and the like. For example, in a case where it is recognized in step S305 that the amplitude of the heart sound signal is relatively small, the guidance on how to deal with the error may include instructing the user to manually adjust the pressure. In addition, in a case where a heart sound signal similar to the reference waveform cannot be acquired a predetermined number of times or more, the control unit 126 may display information indicating that the user should contact the medical institution to readjust the heart sound acquisition system 100A. In that case, the heart sound acquisition system 100A may interrupt the processing in step S306.

After displaying the error, the cause of the error, and the guidance on how to deal with the error to the user in step S306, the control unit 126 waits for the acquisition of the heart sound by the user again. After adjusting the pressure for pressing the heart sound acquisition site of the heart sound sensor 111 according to the guidance or the like, the user acquires the heart sound again (step S303).

When determining in step S304 that the measurement waveform is similar to the reference waveform (Yes in step S304), the control unit 126 measures a heart sound signal for a predetermined time (step S307).

After step S307, the power of the heart sound acquisition system 100A is turned off by manual operation performed by the user or by a program of the system, and the processing ends (step S308).

As described above, the heart sound acquisition system 100A according to the thirteenth embodiment can acquire the heart sound having a waveform close to the reference waveform of the heart sound measured by the medical worker such as a doctor. Therefore, even a user with less medical knowledge can acquire a heart sound waveform close to the heart sound waveform acquired by a specialized medical worker when acquiring a heart sound at home or the like. As a result, reproducibility in acquiring heart sounds is further enhanced.

### Fourteenth Embodiment

A heart sound acquisition system 100B according to the fourteenth embodiment acquires a heart sound using a reference waveform of a heart sound signal acquired in advance by a medical worker such as a doctor, as in the heart sound acquisition system 100A according to the thirteenth embodiment. The heart sound acquisition system 100B uses a heart sound acquisition unit 110 having a controllable pressure adjustment mechanism 113. The heart sound acquisition unit 110 can include, for example, the heart sound acquisition device 50 according to the tenth embodiment and the heart sound acquisition device 60 according to the eleventh embodiment.

The heart sound acquisition system 100B is similar to the heart sound acquisition system 100A according to the thirteenth embodiment, and thus, the same or similar components are denoted by the same reference numerals as those of the heart sound acquisition system 100A, and only a different point from the heart sound acquisition system 100A will be described.

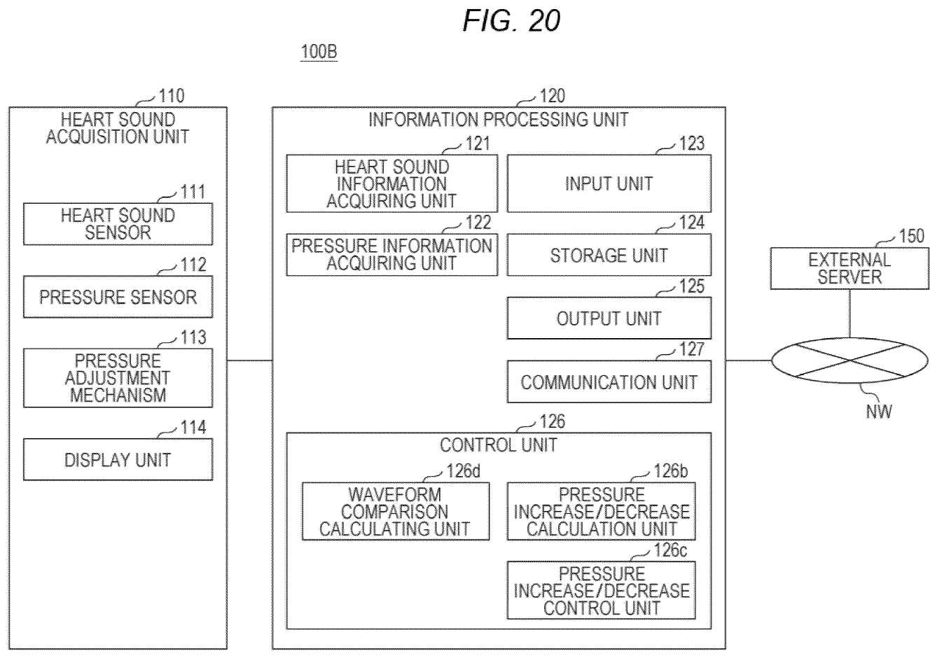

As illustrated in FIG. 20, the heart sound acquisition system 100B is different from the heart sound acquisition system 100A in having a pressure adjustment mechanism 113 as an adjustment portion in the heart sound acquisition unit 110 and the configuration of the control unit 126. The control unit 126 of the heart sound acquisition system 100B includes a pressure increase/decrease calculation unit 126b and a pressure increase/decrease control unit 126c, as in the heart sound acquisition system 100 according to the twelfth embodiment. The control unit 126 of the heart sound acquisition system 100B further includes a waveform comparison calculating unit 126d similar to that in the thirteenth embodiment.

The pressure increase/decrease calculation unit 126b can determine an increase or decrease in pressure applied by the heart sound sensor 111 to press the human body at the time of next acquisition of the heart sound according to a heart sound acquisition state determined by the waveform comparison calculating unit 126d. The pressure increase/decrease calculation unit 126b is further configured to calculate an adjustment amount for increasing or decreasing the pressure.

The pressure increase/decrease control unit 126c is configured to transmit a control signal to the pressure adjustment mechanism 113 of the heart sound acquisition unit 110 on the basis of the result calculated by the pressure increase/decrease calculation unit 126b and adjust the pressure that is applied by the heart sound sensor 111 of the heart sound acquisition unit 110 to press the human body.

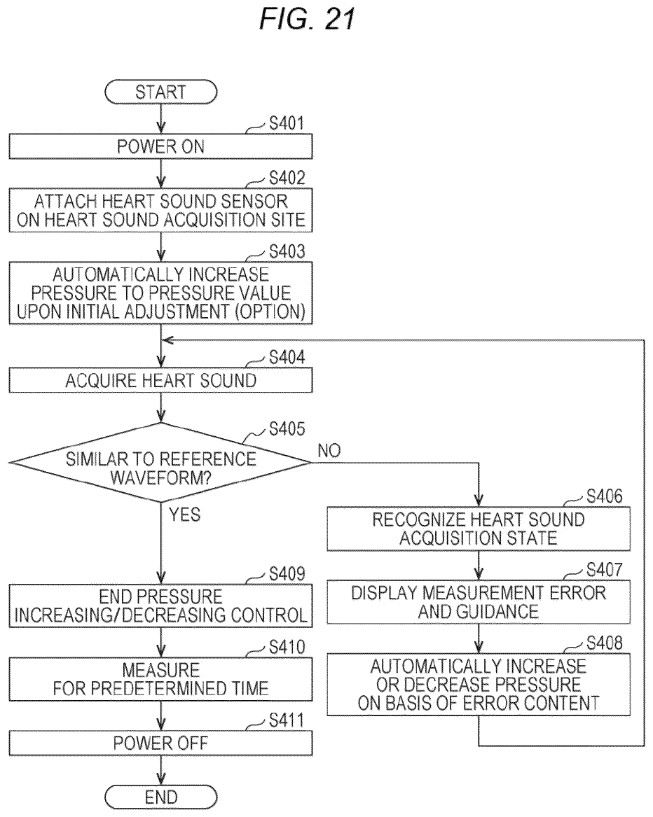

A method for acquiring a heart sound using the heart sound acquisition system 100B according to the fourteenth embodiment will be described below with reference to FIG. 21. It is assumed that the reference waveform of the heart sound signal acquired in advance is stored in the storage unit 124 by a procedure similar to the procedure described with reference to FIG. 18 in the thirteenth embodiment. Furthermore, the pressure measured by the pressure sensor 112 when the reference waveform is acquired may be stored in the storage unit 124 as the reference pressure.

First, the user turns on the heart sound acquisition system 100B and activates the heart sound acquisition system 100B (step S401).

The user attaches the heart sound sensor 111 of the heart sound acquisition unit 110 on the heart sound acquisition site of the subject (step S402). The information processing unit 120 can detect that the heart sound acquisition unit 110 is attached on the subject based on a signal from the heart sound information acquiring unit 121 and/or the pressure information acquiring unit 122.

The control unit 126 (pressure increase/decrease control unit 126c) of the information processing unit 120 controls the pressure adjustment mechanism 113 to automatically increase the pressure applied by the heart sound sensor 111 for pressing the human body to an initially set pressure (step S403). In a case where the storage unit 124 stores the reference pressure, the control unit 126 may control the pressure adjustment mechanism 113 so that the pressure measured from the pressure sensor 112 via the pressure information acquiring unit 122 reaches the reference pressure as the initial value of the pressure.

After step S403, the heart sound acquisition unit 110 acquires the heart sound from the heart sound sensor 111 (step S404). The heart sound signal acquired by the heart sound sensor 111 is transmitted to the information processing unit 120 via the heart sound information acquiring unit 121. As a result, the control unit 126 acquires the heart sound signal.

The control unit 126 (waveform comparison calculating unit 126d) of the information processing unit 120 compares a measurement waveform that is the waveform of the acquired heart sound signal of the subject with the reference waveform stored in the storage unit 124, and determines the similarity between both waveforms (step S405). A method such as DTW described in the thirteenth embodiment can be used to determine the similarity between the waveforms.

When determining that the measurement waveform is not similar to the reference waveform (No in step S405), the control unit 126 recognizes a heart sound acquisition state on the basis of the acquired heart sound signal (step S406). The heart sound acquisition state includes information indicating that the amplitude of the heart sound signal is smaller or larger than the reference waveform by a predetermined percentage or more, or the waveform of the heart sound signal and the reference waveform are significantly different in shape from each other. The control unit 126 may sequentially store the information regarding the amplitude of the heart sound signal in the storage unit 124.

After step S406, the control unit 126 displays an occurrence of a measurement error, a cause of the error, and/or a guidance on how to deal with the error on the output unit 125 and/or the display unit 114 of the heart sound acquisition unit 110 on the basis of the heart sound acquisition state recognized in step S405 (step S407). In the heart sound acquisition system 100B according to the fourteenth embodiment, the pressure is automatically adjusted, and thus, displaying the guidance in step S407 is not always necessary.

The control unit 126 (pressure increase/decrease calculation unit 126b) determines whether to increase or decrease the pressure applied by the heart sound sensor 111 on the basis of the recognized error content (step S408). The control unit 126 may determine to increase or decrease the pressure in a direction in which the amplitude increases on the basis of the accumulated information regarding the amplitude of the heart sound signal in the past. In addition, the control unit 126 may determine the adjustment amount of pressing on the basis of the relationship between the pressing force of the heart sound sensor 111 and the amplitude of the heart sound signal in the past. The pressure increase/decrease control unit 126c of the control unit 126 controls the pressure adjustment mechanism 113 of the heart sound acquisition unit 110 on the basis of the increase or decrease of the pressure determined by the pressure increase/decrease calculation unit 126b.

After step S408, the control unit 126 returns to the process of step S404.

When determining in step S405 that the waveform of the acquired heart sound signal is similar to the reference waveform (Yes in step S405), the control unit 126 ends the control of increasing or decreasing the pressing force applied by the heart sound sensor 111 to the human body using the pressure adjustment mechanism 113 (step S409).

After finishing the process of increasing or decreasing the pressure, the control unit 126 measures the heart sound of the subject for a predetermined time (step S410).

After step S410, the power of the heart sound acquisition system 100B is turned off by manual operation performed by the user or by a program of the system, and the processing ends (step S411).

As described above, the heart sound acquisition system 100B according to the fourteenth embodiment can acquire the heart sound having a waveform close to the reference waveform of the heart sound measured by the medical worker such as a doctor, as in the heart sound acquisition system 100A according to the thirteenth embodiment. Furthermore, the pressure applied by the heart sound sensor 111 to press the heart sound acquisition site of the subject is automatically adjusted by the system, whereby the heart sound acquisition system 100B can perform measurement without relying on the user performing the measurement. Thus, the reproducibility of the acquired heart sound data is further improved, and the heart sound can be acquired with relatively high accuracy.

In the fourteenth embodiment, the pressure sensor 112 of the heart sound acquisition unit 110 and the pressure information acquiring unit 122 of the information processing unit 120 are not always necessary. As described in the heart sound acquisition system 100 according to the twelfth embodiment, the control unit 126 can bring the waveform of the heart sound signal obtained from the heart sound sensor 111 close to the reference waveform while adjusting the pressure adjustment mechanism 113 without data of the pressure value by the pressure sensor 112.

### Fifteenth Embodiment

A heart sound acquisition system 100C according to the fifteenth embodiment of the present disclosure can be used to measure and estimate a parameter related to a cardiac function in combination with another measurement device that measures biological information other than the heart sound in addition to the measurement of the heart sound. Example of the other measurement devices can include at least one of an electrocardiograph, a blood-pressure meter, and a sphygmograph.

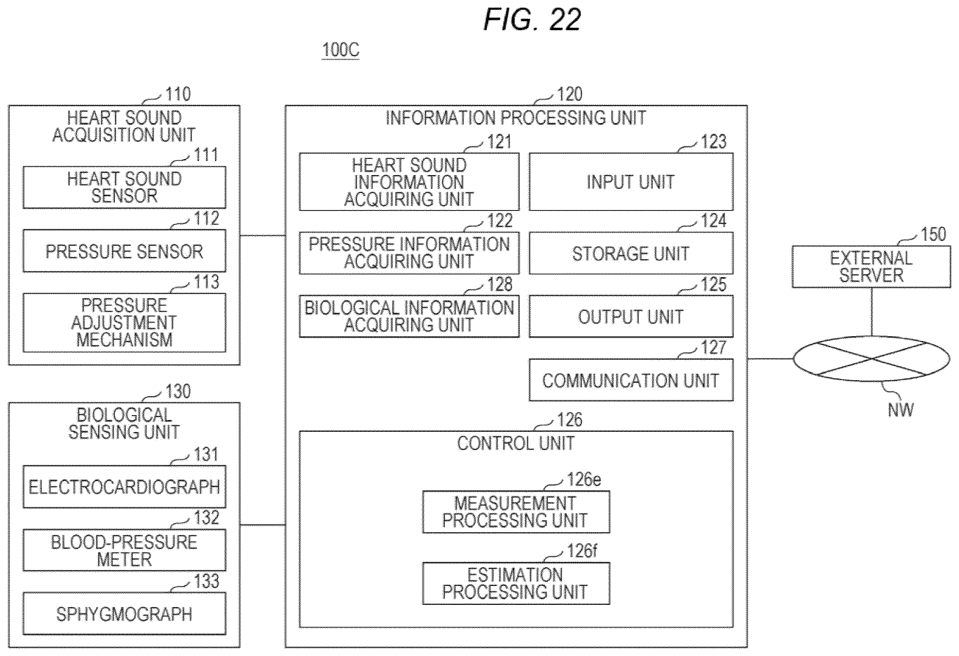

The heart sound acquisition system 100C will be described with reference to FIG. 22. The heart sound acquisition system 100C has portions common to the heart sound acquisition system 100B according to the fourteenth embodiment. In the following description, components same as or similar to those included in the heart sound acquisition system 100B according to the fourteenth embodiment are denoted by the same reference numerals as those of the heart sound acquisition system 100B. The heart sound acquisition system 100C includes a heart sound acquisition unit 110, an information processing unit 120, and a biological sensing unit 130. The heart sound acquisition unit 110 and the information processing unit 120 may be the same device or may be separate devices.

The heart sound acquisition unit 110 includes a heart sound sensor 111, a pressure sensor 112, and a pressure adjustment mechanism 113 which are components similar to the components included in the heart sound acquisition system 100B according to the fourteenth embodiment.

The biological sensing unit 130 detects biological information other than heart sounds. The biological sensing unit 130 includes at least one of a plurality of measurement devices including an electrocardiograph 131, a blood-pressure meter 132, and a sphygmograph 133. The biological sensing unit 130 may be a single device that performs a plurality of types of measurements, or may be a plurality of independent measurement devices. At least a part of the measurement function of the biological sensing unit 130 may be provided using the same device as the heart sound acquisition unit 110. The measurement result of each of the measurement devices of the biological sensing unit 130 may be transmitted to the information processing unit 120.

The information processing unit 120 includes a heart sound information acquiring unit 121, a pressure information acquiring unit 122, an input unit 123, a storage unit 124, an output unit 125, a communication unit 127, and a control unit 126, which are components similar to the components included in the heart sound acquisition system 100B according to the fourteenth embodiment. The information processing unit 120 further includes a biological information acquiring unit 128 that acquires biological information other than heart sounds. The biological information acquiring unit 128 receives an input from the biological sensing unit 130.

The storage unit 124 may store a trained model by machine learning having at least any one of biological information including an electrocardiogram, a blood pressure, and a pulse wave as an explanatory variable in addition to heart sounds and a parameter related to a patient's cardiac function as an objective variable. The trained model can be generated by collecting heart sound data, biological information other than heart sounds, and parameters related to cardiac functions of a large number of patients, and performing machine learning using another computer in advance.

The control unit 126 includes two functional blocks that are a measurement processing unit 126e and an estimation processing unit 126f. The measurement processing unit 126e executes processing of determining a measurement value of the biological information such as the heart sound, the electrocardiogram, the blood pressure, and the pulse wave on the basis of the outputs of the heart sound acquisition unit 110 and the biological sensing unit 130. The functions of the waveform comparison calculating unit 126d, the pressure increase/decrease calculation unit 126b, and the pressure increase/decrease control unit 126c included in the control unit 126 of the heart sound acquisition system 100B according to the fourteenth embodiment are included in the measurement processing unit 126e.

The measurement processing unit 126e processes, besides the heart sound, at least one of biological information including the electrocardiogram, the blood pressure, and the pulse wave acquired from the biological sensing unit 130 via the biological information acquiring unit 128, and determines the measurement value or the measurement waveform. The measurement processing unit 126e combines the heart sound data with other biological information to measure a parameter related to the cardiac function. The parameter related to the cardiac function in this case may include, for example, a systolic time interval (STI) such as a preejection period (PEP) or a left ventricular ejection time (LVET). The measurement processing unit 126e may store the measurement result in the storage unit 124.

The estimation processing unit 126f inputs the heart sound data and the measurement data of the biological information obtained by the measurement processing unit 126e to the trained model stored in the storage unit 124 as explanatory variables, whereby parameters regarding the cardiac function can be estimated by machine learning. The parameter related to the cardiac function is a parameter indicating intracardiac hemodynamics and may include an intracardiac pressure, and the intracardiac pressure may include, for example, any of the left heart pressure, the right heart pressure, a pressure waveform, and the like. The intracardiac pressure can include, for example, left ventricular end-diastolic pressure (LVEDP), pulmonary arterial pressure (PAP), and pulmonary wedge pressure (PWP), but is not limited to the examples of the intracardiac pressures described above. The pulmonary wedge pressure is also referred to as pulmonary arterial wedge pressure (PAWP), pulmonary capillary wedge pressure (PCWP), or pulmonary artery occlusion pressure (PAOP).

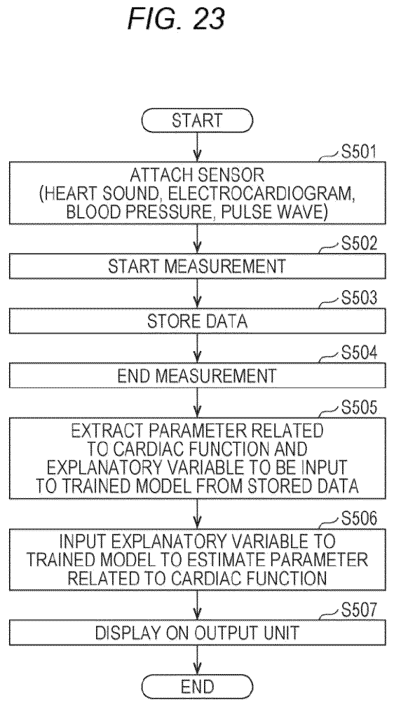

Next, an example of processing of measuring and estimating a parameter related to a cardiac function using the heart sound acquisition system 100C will be described with reference to FIG. 23.

First, in addition to the heart sound acquisition unit 110, the biological sensing unit 130 that measures at least one of the electrocardiogram, the blood pressure, and the pulse wave is attached on the subject (step S501).

Measurement is started by user's operation (step S502). The measurement processing unit 126e of the control unit 126 measures biological information on the basis of output signals from the heart sound acquisition unit 110 and the biological sensing unit 130.

The measurement processing unit 126e of the control unit 126 stores the measured heart sound data and biological information data in the storage unit 124 (step S503). The measurement processing unit 126e may calculate the measurement result by combining two or more pieces of biological information including the heart sound signal.

When the measurement of all the pieces of biological information set in advance is completed, the measurement processing unit 126e of the control unit 126 ends the measurement (step S504).

Next, the estimation processing unit 126f of the control unit 126 extracts an explanatory variable necessary for estimating the parameter related to the cardiac function from the data stored in step S503 (step S505). The extracted explanatory variable is the explanatory variable of the trained model stored in the storage unit 124.

The estimation processing unit 126f of the control unit 126 inputs the explanatory variable of the subject extracted in step S505 to the trained model and estimates the parameter related to the cardiac function (step S506).

The control unit 126 can output the estimated parameter related to the cardiac function to the output unit 125 (step S507) and/or store the parameter in the storage unit 124. The measured and/or estimated parameter related to the cardiac function may be transmitted to the external server 150 via the network NW. The information stored in the external server 150 can be used, for example, as information that enables a medical worker such as a doctor to monitor, in a non-invasive or minimally invasive manner, a hemodynamic parameter indicating the state of the heart of a heart failure patient as a parameter related to the cardiac function, grasp the state of the heart failure patient, and address a change in the state.

As described above, according to the heart sound acquisition system 100C according to the fifteenth embodiment, it is possible to measure and/or estimate the parameter related to the cardiac function in a non-invasive or minimally invasive manner by combining the heart sound information with the biological information acquired from another measurement device while the subject is at home. As in the heart sound acquisition system 100B according to the fourteenth embodiment, heart sound data with high reproducibility can be used, whereby reproducibility and objectivity of measurement and/or estimation of parameters related to the cardiac function are improved.

Although the embodiments according to the present disclosure have been described based on the drawings and examples, it should be noted that those skilled in the art can easily make various modifications or changes based on the present disclosure. Therefore, it should be noted that these modifications or changes fall within the scope of the present disclosure. For example, the shape and arrangement of each component of the heart sound acquisition device described in each embodiment are merely examples. For example, functions and the like of the components, steps, and the like can be rearranged so as not to be logically inconsistent, and a plurality of components, steps, and the like can be combined into one, or divided. Although the embodiments according to the present disclosure have been described mainly with respect to the devices, each of the embodiments according to the present disclosure can also be embodied as a method including steps executed by each of the components of the device. The embodiments according to the present disclosure can also be embodied as methods or programs executed by processors included in the devices or non-transitory computer-readable media recording the programs. It should be understood that these are also included in the scope of the present disclosure.

The detailed description above describes to a heart sound acquisition device, a heart sound acquisition system, a heart sound acquisition method, and a program. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A heart sound acquisition device comprising:
a main body;
a movable member including a first sensor configured to be brought into contact with a human body to acquire a heart sound;
a biasing portion configured to bias the movable member to allow the first sensor to press the human body when the heart sound is acquired; and
wherein the movable member is movable with respect to the main body in a pressing direction that is a direction in which the first sensor presses the human body, and the main body includes an adjustment portion configured to adjust a biasing force of the biasing portion on the movable member.

2. The heart sound acquisition device according to claim 1, wherein the biasing portion is engaged with the main body at a first end along the pressing direction, is engaged with the movable member at a second end closer to the first sensor with respect to the first end, and configured to apply a force in a direction of increasing a distance between the first end and the second end.

3. The heart sound acquisition device according to claim 2, wherein the biasing portion includes at least one of an elastic body or a magnetic body.

4. The heart sound acquisition device according to claim 2, wherein the main body comprises:
a first member; and
a second member that is located on a side of the first member opposite to a side on which the first sensor is mounted, the second member including a first movement restriction portion engaged with the first end of the biasing portion, and the second member includes the adjustment portion configured to adjust a position with respect to the first member in the pressing direction.

5. The heart sound acquisition device according to claim 1, wherein the main body includes a fixing member configured to fix the adjustment portion so as to fix the biasing force.

6. The heart sound acquisition device according to claim 1, wherein the main body includes a second movement restriction portion configured to restrict movement of the movable member in the pressing direction by interfering with the movable member.

7. The heart sound acquisition device according to claim 1, wherein the main body includes a cover configured to come into contact with the human body around the first sensor during acquisition of the heart sound, the cover including the adjustment portion configured to adjust a position with respect to the second movement restriction portion in the pressing direction.

8. The heart sound acquisition device according to claim 7, further comprising:
a second sensor for acquiring biological information other than a heart sound on a surface of the cover to be in contact with the human body.

9. The heart sound acquisition device according to claim 7, further comprising:
an adhesive member that is detachably affixed to the human body on a surface of the cover to be in contact with the human body.

10. The heart sound acquisition device according to claim 1, wherein the main body includes at least one of a protrusion including a sliding surface for movably supporting the movable member in the pressing direction or a ball bearing.

11. The heart sound acquisition device according to claim 1, further comprising:
a mark used for relatively positioning the main body and the movable member in the pressing direction, the mark being attached on at least one of the main body or the movable member.

12. A heart sound acquisition system comprising:
a heart sound acquisition unit including a main body, a movable member including a first sensor configured to be brought into contact with a human body to acquire a heart sound, and a biasing portion configured to bias the movable member to allow the first sensor to press the human body when the heart sound is acquired, the movable member being movable with respect to the main body in a pressing direction that is a direction in which the first sensor presses the human body, the main body including an adjustment portion configured to adjust a biasing force of the biasing portion on the movable member; and
a control unit configured to determine an acquisition state of the heart sound on the basis of data indicating the heart sound acquired from the first sensor.

13. The heart sound acquisition system according to claim 12, further comprising:
a storage unit configured to store a reference waveform of a heart sound measured in advance, wherein the data indicating the heart sound includes a measurement waveform of the heart sound; and
the control unit is configured to determine the acquisition state of the heart sound by comparing the reference waveform with the measurement waveform.

14. The heart sound acquisition system according to claim 13, wherein
the control unit is configured to determine the acquisition state of the heart sound on the basis of similarity between the reference waveform and the measurement waveform;
an output unit, wherein the control unit is configured to cause the output unit to display at least one of an error or a guidance when determining that the reference waveform and the measurement waveform are not similar to each other;
wherein the heart sound acquisition unit includes a third sensor configured to measure a pressure applied by the first sensor to press the human body; and
a storage unit configured to store a reference pressure that is set in advance for acquiring a heart sound, wherein, when the pressure measured by the third sensor is not within a predetermined range based on the reference pressure, the control unit causes the output unit to display at least one of an error or a guidance.

15. The heart sound acquisition system according to claim 13, wherein
the main body includes, as the adjustment portion, a pressure adjustment mechanism configured to adjust pressure applied by the first sensor to press the human body; and
the control unit is configured to adjust the pressure by the pressure adjustment mechanism when determining that the reference waveform and the measurement waveform are not similar to each other.

16. The heart sound acquisition system according to claim 12, wherein
the main body includes, as the adjustment portion, a pressure adjustment mechanism configured to adjust pressure applied by the first sensor to press the human body, the data indicating the heart sound includes a measurement waveform of the heart sound, and the control unit is configured to adjust the pressure by the pressure adjustment mechanism to maximize an amplitude of the measurement waveform;
the heart sound acquisition unit includes a third sensor configured to measure the pressure applied by the first sensor to press the human body; and
a storage unit configured to store a reference pressure that is set in advance for acquiring a heart sound, wherein the control unit is configured to adjust the pressure by the pressure adjustment mechanism to set the pressure measured by the third sensor to the reference pressure as an initial value of the pressure.

17. The heart sound acquisition system according to claim 15, wherein
the biasing portion is engaged with the main body at a first end along the pressing direction, is engaged with the movable member at a second end closer to the first sensor with respect to the first end, and applies a force in a direction of increasing a distance between the first end and the second end, and the pressure adjustment mechanism adjusts a position of the main body engaged with the first end in the pressing direction;
the biasing portion includes a balloon configured to press the movable member in the pressing direction; and
the pressure adjustment mechanism adjusts pressure exerted on the movable member by the balloon by supplying air or discharging air into or from the balloon.

18. The heart sound acquisition system according to claim 12, wherein
the control unit is configured to acquire biological information other than a heart sound from another measurement device, and estimate a cardiac function-related parameter on the basis of the heart sound acquired from the first sensor and the biological information; and
the other measurement device includes at least one of an electrocardiograph or a blood-pressure meter and wherein the cardiac function-related parameter includes an intracardiac pressure.

19. A heart sound acquisition method executed by a control unit of a computer, the method comprising:
acquiring a heart sound from a heart sound acquisition unit that includes a main body, a movable member including a first sensor that is brought into contact with a human body to acquire a heart sound, and a biasing portion that biases the movable member to allow the first sensor to press the human body when the heart sound is acquired, the movable member being movable with respect to the main body in a pressing direction that is a direction in which the first sensor presses the human body, the main body including an adjustment portion that adjusts a biasing force of the biasing portion on the movable member; and
determining an acquisition state of the heart sound on the basis of a measurement waveform of the heart sound.

20. A non-transitory computer-readable storage medium storing a program that causes a processor to execute the method according to claim 19.
